SEQUENCE LISTING (49 sequences)

SEQ ID NO: 1 - DNA, 23 bp, Mus musculus
  misc_feature (1)...(23): mouse centromeric probe, minor satellite DNA sense primer
ggaaaatgataaaaaccacactg

SEQ ID NO: 2 - DNA, 23 bp, Mus musculus
  misc_feature (1)...(23): mouse centromeric probe, minor satellite DNA sense primer
tgtttctcattgtaactcattga

SEQ ID NO: 3 - DNA, 18 bp, Mus musculus
  misc_feature (1)...(18): beta actin forward primer
ctggcacccagcacaatg

SEQ ID NO: 4 - DNA, 17 bp, Mus musculus
  misc_feature (1)...(17): human beta actin antisense primer
agcgaggccaggatgga

SEQ ID NO: 5 - DNA, 24 bp, Mus musculus
  misc_feature (1)...(24): TaqMan probe actin
ccgccgatccacacggagtacttg

SEQ ID NO: 6 - DNA, 30 bp, Mus musculus
  misc_feature (1)...(30): beta actin sense primer, hybridizes to both human and mouse
tgacggggtcacccacactgtgcccatcta

SEQ ID NO: 7 - DNA, 30 bp, Mus musculus
  misc_feature (1)...(30): beta actin antisense primer, hybridizes to both human and mouse
ctagaagcatttgcggtggacgatggaggg

SEQ ID NO: 8 - DNA, 20 bp, Homo sapiens
  misc_feature (1)...(20): forward primer human beta actin
ccgacaggatgcagaaggag

SEQ ID NO: 9 - DNA, 20 bp, Homo sapiens
  misc_feature (1)...(20): reverse primer human beta actin
ggcacgaaggctcatcattc

SEQ ID NO: 10 - DNA, 21 bp, Homo sapiens
  misc_feature (1)...(21): human PCNA sense primer
ggagaacttggaaatggaaac

SEQ ID NO: 11 - DNA, 21 bp, Homo sapiens
  misc_feature (1)...(21): human PCNA antisense primer
ctgcatttagagtcaagaccc

SEQ ID NO: 12 - DNA, 23 bp, Homo sapiens
  misc_feature (1)...(23): human myf5 sense primer
tgagagagcaggtggagaactac

SEQ ID NO: 13 - DNA, 23 bp, Homo sapiens
  misc_feature (1)...(23): human myf5 antisense primer
gccttcttcgtcctgtgtattag

SEQ ID NO: 14 - DNA, 20 bp, Homo sapiens
  misc_feature (1)...(20): human myogenin sense primer
gccacagatgccactacttc

SEQ ID NO: 15 - DNA, 20 bp, Mus musculus
  misc_feature (1)...(20): human myogenin antisense primer
caacttcagcacaggagacc

SEQ ID NO: 16 - DNA, 20 bp, Homo sapiens
  misc_feature (1)...(20): human desmin sense primer
cctactctgccctcaacttc

SEQ ID NO: 17 - DNA, 20 bp, Homo sapiens
  misc_feature (1)...(20): human desmin antisense
agtatcccaacaccctgctc

SEQ ID NO: 18 - DNA, 20 bp, Homo sapiens
  misc_feature (1)...(20): human dystrophin sense primer
cagtagccccatcacatttg

SEQ ID NO: 19 - DNA, 20 bp, Homo sapiens
  misc_feature (1)...(20): human dystrophin antisense
ataacgcaatggacaagtgg

SEQ ID NO: 20 - DNA, 20 bp, Homo sapiens
  misc_feature (1)...(20): SkMactin sense primer
cgtggctactccttcgtgac

SEQ ID NO: 21 - DNA, 20 bp, Homo sapiens
  misc_feature (1)...(20): SkMactin antisense
cccattgagaagattcgtcg

SEQ ID NO: 22 - DNA, 20 bp, Homo sapiens
  misc_feature (1)...(20): MCK sense primer
ggcacaatgacaacaagagc

SEQ ID NO: 23 - DNA, 20 bp, Homo sapiens
  misc_feature (1)...(20): MCK antisense primer
gaaaagaagaggaccctgcc

SEQ ID NO: 24 - DNA, 20 bp, Homo sapiens
  misc_feature (1)...(20): MyHCIIx/d sense primer
ataggaacacccaagccatc

SEQ ID NO: 25 - DNA, 20 bp, Homo sapiens
  misc_feature (1)...(20): MyHCIIx/d antisense primer
tttgcgtagacccttgacag

SEQ ID NO: 26 - DNA, 20 bp, Homo sapiens
  misc_feature (1)...(20): MyHCIIx/d sense primer
tcacactcctcgccctattg

SEQ ID NO: 27 - DNA, 20 bp, Homo sapiens
  misc_feature (1)...(20): osteocalcin antisense primer
gaagaggaaagaagggtgcc

SEQ ID NO: 28 - DNA, 20 bp, Homo sapiens
  misc_feature (1)...(20): alpha 1(IX) collagen sense primer
actgggttctctgggtagcc

SEQ ID NO: 29 - DNA, 20 bp, Homo sapiens
  misc_feature (1)...(20): alpha 1 (IX) collagen antisense primer
atgtgctgatctgtcggtgc

SEQ ID NO: 30 - DNA, 22 bp, Homo sapiens
  misc_feature (1)...(22): aP2 sense primer
tatgaaagaagtaggagtgggc

SEQ ID NO: 31 - DNA, 22 bp, Homo sapiens
  misc_feature (1)...(22): aP2 antisense primer
ccaccaccagtttatcatcctc

SEQ ID NO: 32 - DNA, 22 bp, Homo sapiens
  misc_feature (1)...(22): MGF sense primer
tcttcagttcgtgtgtggagac

SEQ ID NO: 33 - DNA, 20 bp, Homo sapiens
  misc_feature (1)...(20): MGF antisense primer
ttgttggtagatgggggctg

SEQ ID NO: 34 - DNA, 20 bp, Mus musculus
  misc_feature (1)...(20): MGF sense primer
ttcagttcgtgtgtggaccg

SEQ ID NO: 35 - DNA, 20 bp, Mus musculus
  misc_feature (1)...(20): MGF antisense primer
ttgtttgtcgatagggacgg

SEQ ID NO: 36 - DNA, 21 bp, Homo sapiens
  misc_feature (1)...(21): Flk-1 sense primer
tgttgttctttccaccagcag

SEQ ID NO: 37 - DNA, 20 bp, Homo sapiens
  misc_feature (1)...(20): Flk-1 antisense primer
acggtctggaaggaactctc

SEQ ID NO: 38 - DNA, 20 bp, Homo sapiens
  misc_feature (1)...(20): CDMP1 sense primer
gccctgttcctggtgtttgg

SEQ ID NO: 39 - DNA, 20 bp, Homo sapiens
  misc_feature (1)...(20): CDMP1 antisense primer
gctgtgtagatgctcctgcc

SEQ ID NO: 40 - DNA, 20 bp, Homo sapiens
  misc_feature (1)...(20)
acggcatgatggactacagc

SEQ ID NO: 41 - DNA, 20 bp, Homo sapiens
  misc_feature (1)...(20): MyoD antisense primer
cacctgctacatttgggacc

SEQ ID NO: 42 - DNA, 20 bp, Homo sapiens
  misc_feature (1)...(20): c-Kit sense primer
gaggggaaaacaccataagg

SEQ ID NO: 43 - DNA, 20 bp, Homo sapiens
  misc_feature (1)...(20): c-Kit antisense primer
gatgccttccacttcctctg

SEQ ID NO: 44 - DNA, 20 bp, Homo sapiens
  misc_feature (1)...(20): c-Met sense primer
ccaatgtcctctcgctcctg

SEQ ID NO: 45 - DNA, 20 bp, Homo sapiens
  misc_feature (1)...(20): c-Met antisense
agaaggaggctggtcgtgtg

SEQ ID NO: 46 - DNA, 20 bp, Homo sapiens
  misc_feature (1)...(20): human CD44 sense primer
ttggagatggattcgtggtc

SEQ ID NO: 47 - DNA, 20 bp, Homo sapiens
  misc_feature (1)...(20): human CD44 antisense primer
ggactcttggactcttctgg

SEQ ID NO: 48 - DNA, 23 bp, Homo sapiens
  misc_feature (1)...(23): CD90 sense primer
atgaacctggccatcagcatcgc

SEQ ID NO: 49 - DNA, 20 bp, Homo sapiens
  misc_feature (1)...(23): CD90 antisense primer
ctgtgacgttctgggaggag

(19) United States
(12) Patent Application Publication
(10) Pub. No.: US 2005/0281788 A1
(43) Pub. Date: Dec. 22, 2005

(54) Compositions comprising muscle progenitor cells and uses thereof

(76) Inventors: Cosimo De Bari, Kent (GB); Frank Luyten, Kraainem (BE); Francesco Dell'Accio, Kent (GB)

Correspondence Address: CLARK & ELBING LLP, 101 FEDERAL STREET, BOSTON, MA, 02110, US

(21) Appl. No.: 10/522,362

(22) PCT Filed: Jul. 30, 2003
(86) PCT No.: PCT/EP03/09008

§ 371 Date: Jun. 25, 2005

(30) Foreign Application Priority Data
Jul. 30, 2002 (US) 60399745

(51) Int. Cl. A61K 45/00; C12N 5/08

(52) U.S. Cl. 424/93.7; 435/366

(57) ABSTRACT

The present invention shows in vivo myogenic differentiation of muscle progenitor cells (MPCs), being derived from joint tissue, in a mouse model of skeletal muscle regeneration. MPCs participated in the regeneration process by long-term persistence and contribution to the compartment of myonuclei and the pool of functional satellite cells. When injected into dystrophic muscles of immunosuppressed mdx mice, human MPCs restored dystrophin in some fibers, and rescued the expression of mouse mechano-growth factor. In addition, the human MPCs derived from synovial membrane were injected into infarcted myocardial muscle. The MPCs engrafted successfully, underwent proliferation and differentiation leading to functional recovery and maintenance of the cardiac muscle. MPCs represent an alternative source of myogenic cells in therapeutic approaches for postnatal skeletal and cardiac muscle repair.

### FIELD OF THE INVENTION

The present invention relates to the repair or regain of function of muscle especially skeletal or cardiac muscle and the identification of suitable cell types for this purpose as well as quality control methods for selecting cell populations.

### BACKGROUND OF THE INVENTION

Skeletal muscle displays substantial intrinsic repair potential which has been attributed to the persistence of a resident reserve population of undifferentiated mononuclear cells termed ‘satellite cells’. Satellite cells are quiescent in mature skeletal muscle and are activated in response to environmental triggers such as injury to mediate postnatal muscle regeneration. Myoblasts display unique features in vitro, such as the expression of myogenic regulatory factors (MRFs) and the formation of multinucleated myotubes under appropriate conditions (Seale & Rudnicki in Dev Biol (2000) 218, 115-124; Seale et al. in Dev Cell (2001) 1, 333-342). The identification of postnatal progenitor cells has opened new opportunities for cell-based technologies for tissue regeneration. Skeletal myoblasts would represent the natural first choice in cellular therapeutics for skeletal muscle, because of their inherent myogenic commitment.

However, the number of muscle precursor cells decreases with age, and this progressive decline is dramatically accelerated in pathologic conditions such as severe and diffuse traumas (e.g. crush syndrome), dystrophy syndromes, or disuse atrophy. Duchenne muscular Dystrophy (DMD), is an X-linked recessive muscular disease characterized by the absence of dystrophin, which results in destabilization of the muscle cell structure, making muscle fibres susceptible to contraction-associated mechanical stress and degeneration. In the first phase of the disease, new muscle fibers are formed by satellite cells. After depletion of the satellite cell pool in childhood, skeletal muscles degenerate progressively an irreversibly and are replaced by fibrotic tissue (Cossu & Mavilio in J Clin Invest (2000), 105(12), 1669-1674). in diffuse muscle disorders such as DMD, in which the availability of a sufficient amount of functional myoblasts is limited, there is a need for alternative sources of myogenic progenitor cells (Webster & Blau in Somat Cell Mol Genet (1990) 16, 557-565). Skeletal myogenic differentiation in vivo has been described using different cell types, including bone marrow (BM) cells, neural stem cells, liver cells, and dermal fibroblasts (Grounds et al. in J Histochem Cytochem (2002) 50, 589-610.).

BM contains two types of stem cells, the hematopoietic stem cells (HSCs) and the mesenchymal stem cells (MSC). They both have myogenic potential (Ferrari et al. in Science (1998) 279, 1528-1530), and circulate in the bloodstream (Wright et al. in Science (2001) 294, 1933-1936; Kuznetsov et al. in J Cell Biol (2001) 153, 1133-1140), with possible colonization of other tissues including the synovial membrane (SM). The competence of implanted stem cells to contribute to the replenishment of the satellite cell compartment would safeguard the long-term regeneration potential for muscle tissue homeostasis and repair, by preventing the depletion of these cells. However, increasing evidence suggests that MSCs isolated from different tissues and organs may have different phenotypic and biological characteristics in vitro and in vivo (Kuznetsov et al. in J Cell Biol (2001) 153, 1133-1140; Young et al. in Anat Rec (2001) 264, 51-62).

The inventors of the present application reported the isolation and characterization of MSCs from the synovial membrane of adult human donors (De Bari et al. Arthritis Rheum (2001) 44, 1928-1942). Synovial membrane derived mesenchymal stem cells (SM-MSCs) were found to be easily expandable in culture, and to maintain a stable molecular profile and multipotentiality in vitro over 10 passages. However, the potential of in vitro muscle formation of these cells was limited. Studies of limb development indicate that the SM may be embryologically related to the joint interzone. SM has been reported to contain cells expressing Wnt-14, a gene belonging to the family of the Wnts and known to play a central role in initiating synovial joint formation in the chick developing appendicular skeleton (Hartmann et al. in Cell (2001) 104, 341-351). The persistence in postnatal synovial tissue of cells with a phenotype reminiscent of the developing joint interzone (De Bari et al cited supra; Hartman et al. cited supra) points to the SM as a possible reservoir of uncommitted progenitor cells for the repair of those joint tissues, such as articular cartilage and menisci, which have a limited capacity for intrinsic repair (Hunziker & Rosenberg in J Bone Joint Surg Am (1996) 78, 721-733). However, manipulations such as tissue dissection, cell isolation and subsequent culture expansion can profoundly influence patterns of gene expression and differentiation potentials. Experiments performed with isolated cell populations therefore do not necessarily reflect what these cell populations do when left in their physiological environment.

Upon mechanical overload of muscle a protein called Mechano Growth Factor (MGF) is strongly upregulated in normal skeletal muscle (Yang et al cited supra; Goldspink cited supra). MGF is a muscle splice form of IGF-1 (insulin related growth factor I) and is expressed both in skeletal muscle and in cardiac muscle and appears to be a critical factor controlling local muscle repair, maintenance, and remodelling (Yang et al. in J Muscle Res Cell Motil (1996) 17, 487495). In the mdx model of DMD, characterized by an absence of dystrophin in the skeletal muscle fibers, MGF is also not detectable in the MDX mouse muscles (Goldspink in J Anat (1999) 194, 323-334). Probably involved in mechano-transduction mechanisms (Gillis in J Muscle Res Cell Motil (1999) 20, 605-625), dystrophin may play a role in the regulation of MGF expression in muscle fibers in response to mechanical stimuli (Goldspink cited supra). In view of the above, MGF could be regarded as a possible surrogate marker associated with functional muscle repair.

The preceding overview shows that there is a further need for ways to supply or replenish damaged or diseased muscle and for safe and easily available medications which contribute to both direct and persistent muscle repair and/or restore the functional performance of muscle.

### SUMMARY OF THE INVENTION

The present invention relates to progenitor cells for the manufacturing of a medicament/therapeutic product for the promotion of muscle cell formation in vivo, e.g. for the treatment of damaged muscle and/or for the treatment of dystrophic muscle diseases. The invention further relates to muscle specific vehicles for the site-specific delivery of gene products.

Thus, a first object of the present invention is to provide a pharmaceutical preparation for the promotion of muscle cell formation in, vivo, e.g. in the treatment, repair or regain of function of muscle cells, especially without ectopic muscle or tumour formation.

It is a further object of the present invention to provide a pharmaceutical preparation for the treatment, repair or regain of function of diseased muscle cells, especially when the disease is rare, i.e. commercially not justifying a dedicated pharmaceutical.

Still a further object of the present invention is to provide a pharmaceutical preparation for the adjunctive therapy of diseases in which repair of muscle or regain of function of muscle cells would improve recovery, e.g. myocardial infarction.

The present invention presents unexpected in vivo results of a population of synovial membrane derived muscle progenitor cells (SM-MPCs). The MPCs show unique characteristics over existing myogenic precursors with respect to providing a persistent reserve population of cells having the attributes of satellite cells and with respect to their ability to regain the expression of a crucial protein for muscular performance (the IGF-I isoform Mechano Growth Factor (MGF)) in a dystrophic muscle mouse model, the mdx mouse. Delivery of muscle precursors through the bloodstream represents an ideal route for the distribution to all skeletal muscles.

The invention relates to compositions comprising a population of mammalian muscle progenitor cells derived from joint tissue, said cells having in vivo myogenic properties and providing a persistent pool of satellite cells when introduced into mammals. The joint tissue used for the isolation of muscle progenitor cells is a synovial joint (diarthrosis). Preferably the joint tissue used in the present invention for the isolation of cells is the synovial membrane.

In a further aspect of the invention the cells of the compositions express one or more of the synovial fibroblast positive markers CD44 and CD90 and/or express the negative markers flk-1 or any marker coexpressed or codetectable with these positive and/or negative markers. The coexpressed or codectable positive markers should be expressed when CD44 and CD90 are expressed and be not expressed when these are not expressed. The coexpressed or codectatble negative markers should be expressed when flk-1 is expressed and be not expressed when this is not expressed.

In another aspect of the invention the cells or the cell populations of the composition express c-met as a positive marker or any marker coexpressed or codetectable with this positive marker. Such coexpressed or codectable positive markers should be expressed when c-met is expressed and be not expressed when it is not expressed. More particularly, the invention relates to a muscle progenitor cell population substantially enriched for the expression of c-met, wherein at least 80% of the cells express c-met.

In another aspect of the invention the cells or cell populations of the composition express cdmp1 as a negative marker or any marker coexpressed or codetectable with this negative marker. Such coexpressed or codectable negative markers should be expressed when cdmp1 is expressed and be not expressed when it is not expressed.

In another aspect of the invention the cells of the composition are genetically engineered. Optionally, the genetically engineered cells comprise a promoter operably linked to a nucleotide sequence encoding a protein selected from the group of an angiogenic factor, a peptide growth factor and an anti-angiogenic factor.

In another aspect of the invention the cells of the composition are clonal and/or cryopreserved.

In yet another aspect of the invention the cells or cell populations are isolated and passaged, preferably between 3 and 10 passages.

The invention further relates to a pharmaceutical composition comprising muscle progenitor cells in admixture with at least one pharmaceutically acceptable carrier.

The invention also relates to a composition comprising muscle progenitor cells for the manufacture of a medicament for the promotion of muscle cell formation, particularly for the repair or prevention of a muscle dysfunction.

In one particular aspect of the invention the dysfunctional muscle is skeletal muscle and the dysfunction is selected from the group of a severe trauma, a diffuse trauma and crush syndrome, disuse atrophy and sarcopenia.

In another aspect the dysfunction is a muscular dystrophy such as Duchenne Muscular Dystrophy.

In one aspect of the invention the dysfunctional muscle is caused by an ischemic event.

In another aspect of the invention the dysfunctional muscle is cardiac muscle and dysfunction is a cardiovascular disorder selected from at least myocardial infarct and heart failure.

The compositions of the present function can be administered locally or systemically.

The invention also relates to compositions comprising muscle progenitor cells for the manufacture of a medicament for the restoration of MFG expression by dystrophic muscle cells.

The invention also relates to compositions comprising muscle progenitor cells for the manufacture of a medicament which ensures the generation of a persistent population of satellite cells.

The present invention further relates to methods of regenerating skeletal or cardiac muscle comprising the step of administrating a composition comprising muscle precursor cells either by local injection or by administration into the blood stream.

The present invention further relates to methods of obtaining a muscle progenitor cell population suitable for use in the prevention or restoration of muscle dysfunction, which comprises enriching a progenitor cell population obtained from a joint tissue for the expression of c-met.

The present invention further relates to methods of selecting muscle precursor cells comprising the step of simultaneously or subsequently contacting a cell population with a binding substance for one or more of the positive and/or negative markers selected from the group of CD90, CD44, c-Met and CDMP1 or any marker coexpressed or codetectable with these positive and/or negative markers. The binding substance can be an antibody or ligand or a receptor.

The present invention further relates to methods cultivating the muscle progenitor cells in low serum containing medium (less than 10%, preferably less than 5%, more preferably less than 2%) prior to administration to an individual.

The present invention further relates to a method of restoring the capacity of dystrophic muscle cells to express MGF comprising the step of administering muscle progenitor cells to an individual with dystrophic muscle.

The present invention further relates to a method providing a persistent reserve population of satellite cells in an individual comprising the step of administering a composition comprising muscle progenitor cells.

The present invention further provides a vehicle for muscle specific delivery of therapeutic agents using the muscle progenitor cells of the present invention

The present invention further relates to a composition of muscle progenitor cells which, after administration to an individual, can provide a persistent pool of satellite cells which can contribute the generation of new myonuclei during muscle regeneration.

### BRIEF DESCRIPTION OF THE FIGURES

FIG. 1 shows the contribution of human SM-MPC to skeletal muscle regeneration in vivo in accordance with embodiments of the present invention. Panel a displays black staining of human nuclei in murine muscle after in situ hybridisation. Scale bar: 200 μm (micrometre). in panel b brightfield (ALU positive nuclei) and fluorescence (DAPI counterstaining) images were given artificial colors and superimposed. The ALU positive human nuclei are shown as dark spots while the ALU negative, DAPI stained nuclei are shown as lighter spots. The human nuclei represented a minority of the overall number of nuclei detected. Panel c shows staining of human cells expressing LacZ after injection in murine muscle. LacZ expressing cells are indicated by arrows (scale bar: 50 μm (micrometer)). Panel d shows the contribution of human cells to muscle fibers as indicated by Immunohistochemistry for human β2-microglobulin (β32M) (dark staining). Scale bar: 20 μm. Panel e shows results of semiquantitative reverse transcription-polymerase chain reaction (RT-PCR) for human myosin heavy chain type IIx/d (MyHC-IIx/d) after injection of human keratinocytes (lane 2), human MPC (lane 3) and human skeletal muscle cells (lane 5). Lane 1 and 4 are controls. Panel f shows a immunofluorescence microphotograph of a double genomic in situ hybridisation on a section from a tibialis anterior (TA) muscle 4 weeks after human SM-MPC transplantation. Human cells probed for chromosome 18 centromeres (dots in the cells) are indicated by arrows. Scale bar: 50 μm.

FIG. 2 shows the in vivo myogenic potential of human SM-MPCs regardless of donor age or cryopreservation in accordance with an embodiment of the present invention. Panel a shows RT-PCR analysis for the expression of human MyHC-IIx/d on human SM-MPC cells before implantation (−) and TA muscles 4 weeks after SM-MPC implantation (+). Cryopreserved cells are indicated by an asterisk. Panel b shows in vivo myogenic potential of human clonalSM-MPCs. Semiquantitative RT-PCR for human MyHC-IIx/d was performed on cells in monolayer before injection (M) and on injected TA muscles (I). Panel c shows the detection of human nuclei in mice injected with SM-MPC with in situ hybridisation for human ALU genomic repeats. Scale bar is 50 μm.

FIG. 3 shows that the differentiation of injected SM-MPC cells into TA muscles of nude mice recapitulates embryonic myogenesis in accordance with an embodiment of the present invention. Human SM-MPCs were injected into regenerating snake venom cardiotoxin (CTX) treated TA muscles of nude mice. Dissected muscle samples were assayed by RT PCR for the presence of embryonic markers.

FIG. 4 shows the contribution of injected SM-MPC to the compartment of functional satellite cells 6 months after injection in accordance with an embodiment of the present invention. Arrows in panel a show human monuclear cells (white staining of human β2M) between murine myofibers (gray staining of murine laminin) Scale bar: 50 μm. Panel b shows a transmission electron microphotograph of a human SM-MPC-derived satellite cell. The arrows indicate the plasma membrane, the arrowhead shows human β2M staining. The basal lamina is indicated by an asterisk. The inset shows an inverted, high-magnified view of the silver grains of the staining for human β2M. Scale bar: 100 μm. Panel c shows the expression of human Myf5 and human PCNA in normal and CTX damaged muscle injected with or without SM-MPC. Panel d shows that human mononuclear cells, recovered from first recipient mice, retain in vivo myogenic activity when transplanted into a second recipient.

FIG. 5 shows that systemically delivered SM-MPC have a preferential homing to damaged muscle in accordance with an embodiment of the present invention. Panel a shows the presence of human cells in damaged muscle after three weeks while the human cells are detected only after 8 weeks in undamaged muscle. Black spots in panel b show human ALU specific staining in CTX treated muscle. The circle in the middle of the inset shows a human nucleus. Scale bar: 100 μm. Panel c shows that human SM-MPC are found in damaged and undamaged muscle and also in lung after 6 months (RT PCR with human beta actin) human MyHC-IIx/d was not detectable in lungs. Panel d shows that local implantation of human SM-MPC into muscles does not lead to heterotropic tissue formation. RT PCR was performed with markers for mature non-muscle mesenchymal lineages, namely aP2 (fatty acid-binding protein aP2) for adipose tissue; OC (osteocalcin) for bone, and Col9 (type IX collagen) for cartilage). Positive controls were human skeletal muscle for MyHC-IIx/d (lane 7), human primary articular chondrocytes for collagen type IX, human trabecular osteoblasts for osteocalcin, human fat tissue for aP2. Panel e displays that subcutaneous injection of SM-MPC does not lead to ectopic muscle formation in the skin. 3 month after injection no human MyHC-IIx/d is detected in skin. Panel f shows the homing pattern of human synovial membrane-derived mesenchymal stem cells, 3 weeks after systemic delivery (5×10⁶ viable cells) in the tail vein of a nude mouse, as determined by semiquantitative RT-PCR using primers specific for human beta-actin. cDNA templates were equalized for mouse/human beta-actin expression. lane 1: right TA (cardiotoxin-injured); 2: left TA (Phosphate Buffered Saline (PBS)-injected); 3: bone; 4: spleen; 5: liver; 6: lungs; 7: heart; 8: brain; 9: rib cartilage; 10: knee joint; 11: bone marrow; 12: Water negative control. Panel g shows in situ hybridization for human-specific ALU genomic repeats on a frozen section from the heart of a nude mouse, 6 months after systemic injection of 5×106 viable culture-expanded human synovial membrane-derived mesenchymal stem cells. The arrow indicates a dark stained human nucleus.

FIG. 6 shows the restoration of mouse MGF expression in mdx dystrophic mice by human SM-MPC in accordance with an embodiment of the present invention. Panel a shows that after injection of SM-MPC in mdx mice human dystrophin is expressed. Panel b shows a network of human dystrophin antibody staining. In panel c, staining for human Alu repeats shows that the dystrophin expressing cells are of human origin. Panel d shows the percentage of centronucleated myofibers obtained with SM-MPC versus PBS injection obtained from three different experiments. Panel e shows the expression of murine MGF after injection of human SM-MPC in mdx mice (semiquantitative RT-PCR) for mouse MGF. Panels f and g show the maximal numbers of respictively human dystophin-positive myofibers and centronucleated dystrophin positive-myofibers in TA muscles of immunosuppressed mdx mice after injected with either human SM-MPCs or pCMV-human full-length dystrophin plasmid without or with Electrotransfer (ET). TA muscles were examined by immunostaining serial transverse cryostat sections for human dystrophin. Data are mean±standard deviation of maximal number of dystrophin-expressing myofibers per muscle. Panel h shows quantitative RT-PCR for mouse MGF. The expression levels of mouse MGF in mdx TA muscles injected with human SM-MPCs were significantly (p<0.05) higher than those found in mdx TA muscles injected with pCMV-dystrophin (with or without electrotransfer), with PBS, or with pCMV-LacZ.

Definitions

A “muscle dysfunction” as used herein refers to any condition whereby the normal function of the muscle concerned is disrupted. A muscle defect can be the result of a physical injury and/or an ischemic event or can be caused by genetic or environmental factors.

“muscular dystrophy” in the present invention refers to myogenic disorders characterised by progressive muscle wasting and weakness of variable distribution and severity.

“Inherited muscular dystrophies” are classified into six major forms based on the distribution of predominant muscle weakness and a seventh group of congenital dystrophy with a more generalized weakness (reviewed in Emery (2002) Lancet 359, 687-695). A first group comprises the dystrophies of the Duchenne and Becker type both caused by mutations of the dystrophin gene. A second group comprises the dystrophies of the Emery Dreyfuss type. The X-linked form is caused by mutations of the STA gene encoding emerin. The autosomal dominant form is caused by mutations of the LMNA gene encoding laminin A and C. A third group comprises the distal muscular dystrophies including Welander's diseases. Apart from one type caused by mutations in the dysferlin gene, the underlying cause is unknown. A fourth group comprises facioscapulohumeral dystrophies associated with a subtelemoric deletion of chromosome 4q. A fifth group comprises oculopharyngeal muscular dystrophies and are associated with prolonged expansions of a GCG repeat in the Poly(A)binding protein (PAB2). Of the sixth group, limb-girdle dystrophies, 15 genetically different types have been identified and are associated with mutations in genes such as Calpain-3, Dysferlin, alpha-, beta-, gamma-, and delta-sarcoglycan, telethonin and Fukutin related protein. The seventh group of congenital disorders is associated with mutations in genes such as merosin, alpha7 integrin, Fukutin, Selenoprotein N1, and glycosyltransferase.

“Mechano Growth Factor” (abbreviated as MGF) relates to an isoform of Insulin related growth factor 1 (IGF-1). MGF is also known as IGF-I Ec in human and IGF-I Eb in rodents. The MGF isoform lacks exons 1 and 2 and 5 and has an insertion of 49-52 nucleotides (depending upon species) between exon 4 and 6 leading to a frameshift and a modifed C amino acid terminal sequence with respect to other IGF splice variants. MGF is only markedly upregulated in exercised and damaged muscle. MGF is distinct from IGF-I Ea also known by the synonyms muscle IGF and muscle-liver type IGF-I.

“Precursor cell” is a cell having the capacity of undergoing differentiation of performing a specific post natal function.

“Muscle progenitor cells” abbreviated as “MPCs” as used herein refers to a cell population which is characterised by the expression of c-MET and CD34 as positive markers and the absence of expression GDF5/CDMP1 as negative markers. The cell population is further identified by its ability to generate skeletal muscle after local or systemic injection into a nude mouse with induced muscular damage. The cell population is also further identified by the capacity of providing a persistent pool of satellite cells after administration to an individual mammal. According to a particular embodiment of the present invention, the muscle progenitor cell populations are obtained from synovial membrane tissue and are referred to as ‘SM-MPCs’.

“Satellite cells” are a reserve population of undifferentiated mononuclear cells, which lie beneath the basal lamina, applied to the sarcolemma of myofibers. Satellite cells are largely responsible for the production of new myonuclei during postnatal muscle growth and regeneration. Satellite cells are characterised by the following specific ultrastructural criteria: a plasma membrane separating the satellite cell from its adjacent muscle fiber, an overlying basal lamina continuous with the satellite cell and associated fiber, and the heterochromatic appearance of the nucleus (Bischoff in, Engel & Franszini-Armstrong, Eds. Myogenesis. New York, McGraw-Hill, (1994), 97-118).

“Persistent” in the present invention means being still present after local or systemic injection after at least 3 months, preferably after 6 months, even more preferably after 9 months, and most preferably after 12 months. The persistent cells may be functional to repair muscle.

A joint as used herein is a union between two or more or more parts of the skeleton, typically bone, but also cartilages earlier in development. A synovial joint (diarthrosis) is one that has a joint cavity that is enclosed by a fibrous capsule linking the skeletal elements. The capsule is lined by a synovial membrane that secretes lubricating and nutritive fluid. Not all joints are synovial. Synovial joints are typical of limbs. Non-synovial joints are called synarthroses and include fibrous joints where skeletal elements are joined by fibrous material (e.g. sutures between bones in the skull cap) and also include cartilaginous joints where two bones are linked by cartilage (e.g. joints between vertebral bodies).

A “marker” as used herein refers to an expressed DNA sequence, for which expression is associated with a trait, characteristic or function. The markers of the present invention are sequences for which expression is associated with the ability to provide, in vivo, a persistent reserve population of cells having the attributes of satellite cells. Moreover the markers of the present invention are associated with the ability to regain the expression of MGF. Thus, there is a difference in expression of these markers between cell populations that are capable of restoring muscle function, by functioning as a reserve population of satellite cells and populations that are not. Preferably this positive or negative correlation is maintained upon subsequent passaging of the cells. According to the present invention, markers are preferentially detected at the mRNA level, using RT PCR (as described in the examples) or other methods known in the art. Quantitative determination of cells expressing a marker protein can be performed with FACS analysis or in situ immune staining. However, the present invention also envisages other detection methods, e.g. at the protein level. For instance, cell populations expressing the cell-surface receptor c-met as a positive marker can be identified using immunological methods.

A cell population expressing a marker refers to a population wherein each marker independently is expressed by at least 50%, preferably at least 75%, more preferably at least 80%, and even more preferably at least 90% of the cells in that population. Thus, according to the present invention an MPC population is a population wherein the markers positively linked to muscle repair are expressed by at least 50% of the cell population.

“Co-expression and co-detectability”: With co-expression, in the context of the present invention, is meant that a second factor or marker is expressed or detectable whenever a first factor or marker is expressed or detectable. Preferably, the second marker is only expressed or detectable when the first marker is expressed or detectable. Such co-expressed or co-detectable factors or markers can be a recognizable cell surface markers, detectable via polyclonal or monoclonal antibodies and/or specific ligands.

“Marker protein”: A polypeptide that distinguishes one cell (or set of cells) from another cell (or set of cells) in a population of cells. For example, a polypeptide that is expressed (either naturally or artificially, e.g. introduced by genetic engineering) on the surface of skeletal precursor cells but not other cells of a cell population serves as a marker protein for the skeletal precursor cells. Typically, the marker protein is a cell-surface antigen, like for instance a growth hormone receptor, such that antibodies that bind the marker protein can be used in cell sorting methods, e.g., to produce a population of cells enriched for cells that express the marker protein. Alternatively, intracellular proteins can be used as marker proteins. For example, fluorescent or luminescent proteins, such as green fluorescent protein e.g. aequorin (green fluorescent protein of Aequoria victoria, Tanahashi et al (1990), Gene 96: 249-255) can be used as the marker protein and can facilitate cell sorting, e.g., by FACS. Also enzymes can be used, provided that the activity of the enzyme can be detected. For example, β-galactosidase (beta-galactosidase) is well suited for use as a marker protein; this enzyme can be detected by introducing into the cell a substrate(s) that release a fluorescent product(s) upon cleavage by the enzyme (available from, e.g., Molecular Probes). Another suitable enzyme is catechol 2,3-dioxygenase, which is encoded by xy/E of Pseudomonas putida (Domen et al (1986), Anal. Biochem. 155, 379-384). The DNA encoding such a marker protein can typically be linked to the regulatory regions of the markers identified for a cell population, so that expression of the marker is easily quantified by the marker protein.

### DETAILED DESCRIPTION OF THE INVENTION

The present invention will be described with reference to certain embodiments and figures but the skilled person will appreciate that these are merely examples of the present invention and that the teaching of this invention may find wide application.

In a first aspect the present invention relates to muscle progenitor cells (MPCs). The cells are mammalian cells. Preferably they are human cells but they can also be cells from animals of commercial interest such as horses, cattle, dogs, pigs and they can also be cells from animals of scientific interest such as monkeys, goats, rabbits, rodents. The MPCs can be obtained as well from juvenile individuals as from adults without age restriction. The MPCs of the present invention are obtained from connective tissue, preferably of the joint (for example synovial fluid) and are more preferably obtained from the synovial membrane of a joint.

The MPCs of the present invention are characterised by the expression of the positive marker c-met and the absence of expression of the negative markers gdf5/cdmp1. Apart from the said markers, optionally additional positive markers such as CD34 and synovial fibroblast-like cell markers such as CD44 and CD90 can be used to isolate and characterise the SM-MPCs of a tissue. The invention further provides MPC populations substantially enriched for expression of c-met, whereby expression of c-met is present in at least 80% of the cells. The invention also includes the identification of a set of molecular markers linked to the outcome of injection or implantation of MPC's in muscle formation. For instance, freshly isolated human or animal MPC's were used for RNA purification and cultivated in vitro. Upon passaging, an aliquot of cells was used for RNA purification, 2 aliquots of cells were injected into the relevant is human or animal patients and examined for muscle formation and the rest replated. RNAs were tested by semi-quantitative RT-PCR for co-expression of genes with c-met.

The MPCs are further functionally characterised by their ability to contribute to the formation of muscle. This muscle can be skeletal muscle but can be also cardiac muscle. The muscle formation can be obtained by local delivery of the MPCs into a muscle as well as by systemic delivery of the cells into the blood stream. The MPCs of the present invention can be both cells which have been expanded or passaged after isolation. Preferably the MPCs of the present invention have been passaged between 3 and 10 passages, although MPCs which have been passaged for more than 10, more than 15, or more than 20 passages are within the scope of the invention as long as they have in vivo myogenic properties. Similarly non-passaged cells or cells which have been passaged once or twice are within the scope of the invention as long as they have in vivo myogenic properties. The present invention also relates to cells which have been stored by cryopreservation. Further the MPCs of the present invention can be a clonal population of cells.

The MCPs of the present invention can be cultivated without addition of externally added growth factors. Alternatively cells can be grown in the presence of supplemented growth factors (such as BMP or TGF) or growth factors can be added to the cell population prior to administration to an individual with a muscle defect.

The present invention also relates to MPCs which have been genetically engineered by the introduction of one or more genes operably linked to a promoter. Vectors and protocols for transfecting eukaryotic cells such as MPCs are known to the skilled person. A non-limiting number of vectors include replication-defective viral vectors, DNA virus or RNA virus (retrovirus) vectors, such as adenovirus, herpes simplex virus and adeno-associated viral vectors. The genes being introduced into MPCs can be either markergenes or genes with therapeutic properties. Examples of genes with therapeutic properties for muscle specific delivery are angiogenic factors such as VEGF and VEGF-related molecules, anti-angiogenic factors (for tumours), peptide growth factors such as IGF-1, Hepatocyte growth factor, GDF 8 inhibitors such as Noggin and (soluble) dominant negative receptors for GDF-8, therapeutic proteins for the treatment of osteoporosis such as PTH, BMPs.

The engineered cells according to the present invention can be used as a muscle specific vehicle for the directed delivery of gene products.

One embodiment of the invention relates to pharmaceutical compositions comprising the MPCs of the present invention and the use of MPCs for the manufacture of a medicament for muscular disorders, dysfunctions or traumas.

For injectable administration, the pharmacutical composition is in sterile solution or suspension or can be resuspended in pharmaceutical- and physiologically-acceptable aqueous or oleaginous vehicles, which may contain preservatives, stabilizers, and material for rendering the solution or suspension isotonic with body fluids (i. e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

For systemic injections in a human being with a blood volume of about 5 liter an amount between about 5×10⁶ to 5×10¹⁰ cells are used, preferably about 5×10⁸ cells are used. For injection into a muscle 5×10⁷ to 5×10¹¹ cells are used, preferably about 5×10⁹ cells are used.

The pharmaceutical composition comprising MPCs can be applied to an individual by systemic injection, whereby the cells migrate to sites of muscular damage by the process of homing. Alternatively or in addition the cells can be administered to the place of muscle damage by injection or by a catheterisation for example in the case of cardiac muscle damage.

The pharmaceutical composition comprising MPCs can be used for the treatment of a trauma or disorder but can also be applied prior to surgical procedures or situations of extreme muscular performance. The cells being used in the pharmaceutical composition are preferably autogeneic although the use of allogeneic cells is not excluded. In this case, an appropriate donor should be used for isolation of the cells, and/or adequate immunosuppressant should be supplied to the recipient of the cells.

In another embodiment, the compositions of MPCs may be used in animal husbandry.

Another embodiment is the use of the MPCs of the present invention for the manufacture of a medicament for the treatment or prevention of muscular disorders or traumas.

A first group of muscle disorders relates to disorders such as severe and diffuse trauma (crush syndrome), disuse atrophy, muscle degradation in elderly people (sarcopenia) and other weaknesses or dysfunctions caused by injury, disease, inactivity, or anoxia- or surgery-induced trauma.

A second group of disorders to be treated with the cells of the present invention relates to muscular dystrophies. The use of MPCs in the treatment of dystrophies is shown in the examples where MPCs induce the expression of MGF, a protein which is upregulated in damaged and stressed skeletal and cardiac muscle. Dystrophies which can be treated are for example Duchenne Muscular Dystrophy (DMD) and Beckers muscular dystrophy, but also other dystrophies. The MPCs are especially suited for treating dystrophies when these cell are transfected with a gene encoding for a wild type version of a gene which is mutated or missing in a dystrophy.

Another group of disorders to be treated by the cells of the present invention relates to cardiovascular disorders. Examples of such cardiovascular disorders are heart failure, or injury associated with myocardial infarction or any condition of localized cardiac muscle injury.

Another aspect of the invention is related to the capacity of the MPCs of the present invention to provide a persistent pool of satellite cells, which can contribute the generation of new myonuclei during muscle regeneration.

In another aspect the invention is related to methods for the isolation or selection of MPC population from a tissue source such synovial membrane. A method for isolating a MPC population from synovial membrane is described in example 1 of the present invention. In addition to this isolation procedure MPCs can be further purified by contacting cells with receptor ligands or antibodies to positive and negative markers expressed by MPCs. More particularly, a c-met positive population isolated from joint tissue according to the present invention can be enriched for the expression of c-met using anti-c-met antibodies or by way of its ligand, hepatocyte growth factor (HGF).

In another aspect the invention is related to the use of positive and negative markers for the quality control of a pool of SM-MPCs prior to delivery in a patient. More specifically, c-met an other markers which are expressed or co-detectable with c-met and therefore predict c-met expression, can be used for quality control and fall within the scope of the present invention. The molecular marker expression can be detected at the mRNA level (e.g., via RT-PCR), at the protein level (e.g. via specific antibodies—polyclonal or monoclonal or via specific ligands (e.g. hepatocyte growth factor as a ligand of c-met). Fluorochrome-labelled ligand or antibody can be used to select the cells expressing the marker via FACS or FISH-FACS or ligand/antibody-coated magnetic beads can be used to sort c-met expressing cells via a magnetic field (Dynabeads).

DNA chips (or genosensors) are miniature arrays of surface-tethered (c)DNA probes (typically oligonucleotides but also longer DNA probes) to which a nucleic acid sample (the “target” sequence) is hybridized. In the context of the present invention, DNA chips can be used as diagnostic tools to rapidly conclude on the suitability of cells such as MPC's to promote the formation of muscle cells. The aim is to produce digital hybridization fingerprints that can be interpreted by computer and for which ratios of “positive” and “negative” markers can be generated. Genosensors can harbour hundreds to thousands (e.g., 12.000) of DNA probes, useful for high throughput DNA marker analysis and messenger RNA profiling (differential display on a chip). Alternatively, smaller sets of probes, duplicated in subarrays across the chip, can be used to interrogate numerous samples in parallel. Oligonucleotides are either synthesized in situ on the support surface of the DNA chip (in situ attachment strategy), or, alternatively, presynthesized oligonucleotides are attached to each site in the array (post-synthesis attachment strategy). The phosphoramidite method of solid phase chemical synthesis is used to generate the oligonucleotides in both cases (Matteuci and Caruthers (1981), J Am Chem Soc 103: 3185-91). The post-synthesis attachment strategy is easy to implement using commercially available equipment and materials (Beattie, In Caetano-Anolles, Gresshoff (ed), DNA Markers. Protocols, applications and overviews. Wiley-VCH, New York, p213-224). More advanced options are available for preparation of higher density arrays (Microfab technologies Inc.: Eggers et al, (1994), BioTechniques 17: 516-525; Accelerator Technology Corp.: McIntyre (1996), IBC Conference on Biochip Array Technologies, Marina del Rey, Calif.; Mirzabekov group: Yershov et al (1996), Proc Natl Acad Sci USA 93: 4913-4918; Khrapko et al (1991), FEBS lett 256: 118-122; Mirzabekov (1994), Trends Biotechnol 12: 27-32). Support surfaces comprise glass, such as microscopy slides, and microchannel glass (Tonucci et al (1992), Science 258: 783-785) or porous silicon (Lehmann (1993), J Electrochem Soc 140: 2836-2843) for use in a flowthrough genosensor (Beatti et al, (1995), Clin Chem 41: 700-706). In the latter, hybridization occurs within three-dimensional volumes, providing an approximately 100-fold greater surface area per unit cross section compared with two-dimensional flat surface designs, greatly increasing thereby the binding capacity per hybridization cell and providing an improved detection sensitivity etc. (Doktycz and Beattie (1996), in: Beugelsdiik A (ed), Automated Technologies for Genome Characterzation. John Wiley & Sons, New York; Beattie (1996), In: Sayler G S (ed), Biotechnology in the Sustainable Environment. Plenum Publishing Corp, New York; Beattie et al (1996), In: Schlegel J (ed), Pharmacogenetics: Bridging the Gap between Basic Science and Clinical Application. IBC Biomedical Library, Southborough, Mass. Oligonucleotide probes are covalently linked to, e.g., silicon dioxide surfaces by applying the methods of Lamture et al (1994), Nucleic Acid Res 22: 2121-2125; Beattie et al (1995), Clin Chem 41: 700-706, Mol Biotechnol 4: 213-225; Doktycz and Beattie (1996), In: Beugelsdiik A (ed), Automated Technologies for Genome Characterization. John Wiley & Sons, New York; Beattie (1996), In: Sayler G S (ed), Biotechnology in the Sustainable Environment. Plenum Publishing Corp, New York; or Beattie et al (1996), In: Schlegel J (ed), Pharmacogenetics: Bridging the Gap between Basic Science and Clinical Application. IBC Biomedical Library, Southborough, Mass. Protocols for attachment to glass surfaces, using 3′-propanolamine oligonucleotids (Genosys Biotechnologies, The Woodlands, Tex.) and to microscopy slides are available from Beattie (Caetano-Anolles, Gresshoff (ed), DNA Markers. Protocols, applications and overviews. Wiley-VCH, New York, p213-224) and Beattie et al (1995), Mol Biotechnol 4: 213-225. A robotic fluid dispensing system is commercially available (e.g. Hamilton Microlab 2200 system equipped with 21 G needles and 50 μl syringes), capable of robotically dispensing droplets as small as 10 nL onto glass slides at 1 mm center-to-center spacing (Beattie et al (1995), Clin Chem 41: 700-706, Mol Biotechnol 4: 213-225).

Genosensors and diagnostics in accordance with the present invention may be used to diagnose the state of cells and cell cultures but may also be used in situ to determine the vitality of human or animal MPC's.

### EXAMPLES

### Example 1

### Contribution of SM-MPC to Myonuclei

Methods

Animal experimentation protocols were approved by the local ethics committee. Eight-to 10-week-old female NMRI nu^{-/-} mice were used for the in vivo model of skeletal muscle regeneration. Immunodeficient mice were chosen to avoid immune rejection of the xenogeneic human cells. Animals were maintained in isolator cages, under pathogen-free conditions. To study myogenic differentiation of human SM-MPCs in vivo, a well-defined model of skeletal muscle injury was adopted, known to result in a rapid regeneration of myofibers (Ferrari et al.cited supra, ‘mdx-model’). The model consists of massively damaging the tibialis anterior (TA) muscle by injecting the snake venom cardiotoxin (CTX). Mice were anesthetized as described in (Raymackers et al. in J Physiol (2000) 527, 355-364), and 25 μl of 10 mM CTX (Latoxan, Hosans, France) were injected in the TA muscle. Twenty-four hours later, 5×10⁵ viable SM-MPCs suspended in 25 μl PBS were transplanted (single-point injection) into the same TA muscle. Cell viability of the injected is cells, as assessed by trypan blue staining, was higher than 98% in all experiments performed.

For all experiments, human SM-MPCs were used between passages 3 and 10 (De Bari et al. cited supra). SM-MPCs were obtained from random biopsies of SM (wet weight 10-50 mg) of the knee joints of human donors of various age (mean 48 years, range 18-83 years) either postmortem within 12 hours of death, or at the time of surgical knee replacement for degenerative osteoarthritis after informed consent was given. MPCs were isolated and expanded in monolayer on plastic in high-glucose DMEM (Dulbecco's modified Eagle's medium, Life Technologies, Merelbeke, Belgium) containing 10% FBS (fetal bovine serum, BioWhittaker, Verviers, Belgium) and antibiotics (100 units/ml penicillin, 100 μg/ml streptomycin, and 0.25 μg/ml amphotericin B, Life Technologies) at 37° C. in a humidified atmosphere of 5% CO₂, as described in De Bari et al. cited supra. At passage 3 (P3), aliquots of cells in DMEM with 20% FBS and 10% DMSO (dimethylsulfoxide, Sigma, Bornem, Belgium) were cryopeserved in liquid nitrogen, thawed after variable times (range 3-36 months), replated and expanded. Cells were cultivated without addition of externally added growth factors. ISH (In situ hybridisation) for human-specific ALU genomic repeats was performed according to Dell'Accio et al. in Arthritis Rheum (2001) 44, 1608-1619. For frozen sections proteinase K (Sigma) treatment at 37° C. was shortened to 10 minutes compared to fresh tissue. An additional stringency wash was performed for 30 minutes at 50° C. in 1×SSC. Slides were mounted in Mowiol containing DAPI for nuclear counterstaining.

Adenovirus vectors and cell transduction: Replication-deficient recombinant adenovirus carrying the bacterial β-gal reporter gene LacZ under the control of cytomegalovirus immediate-early promoter (CMV), and the empty backbone adenovirus were obtained from The Center for Transgene Technology and Gene Therapy, (Leuven, Belgium). For transduction, cells were washed twice with calcium and magnesium-free phosphate buffered saline (PBS), detached from plastic by treatment with 0.25% trypsin containing 1 mM EDTA (Life Technologies), and replated in growth medium after addition of the virus at the concentration of 10 multiplicity of infection (MOI). The next day, the virus supernatant was removed, and the cells were washed with several changes of medium. Five days later, cells were harvested for the in vivo myogenesis assay. The efficiency of transduction, as determined by β-gal staining on cells before transplantation, was about 50%.

Tissue processing: Mice were killed by cervical dislocation at various time-points, according to the experimental protocols. For total RNA extraction, TA muscles were homogenized in TRIzol (Life Technologies). For histology, histochemistry, and ISH, unless differently stated, TA muscles were dissected, and either were fixed overnight at 4° C. in 10% neutral buffered formalin, embedded in paraffin, and sectioned at 5 μm, or were frozen in isopentane-chilled in liquid nitrogen, and sectioned at 10 μm. TA muscles from mdx mice were transversely divided in 2 equal parts, of which one was used for total RNA extraction and the other to make frozen sections for histochemistry. For staining for human β2M (beta 2 microglobulin), specimens were fixed with 2% glutaraldehyde in 0.05 M cacodylate buffer (pH 7.3) at 4° C. for 60 minutes, embedded in paraffin, and sectioned serially at 7 μm thickness. Sections were mounted on poly-L-lysine coated glass slides and Thermanox coverslips (Electron Microscopy Sciences, Fort Washington, Pa.) for light microscopy and TEM, respectively.

Histochemistry: Whole mount staining for β-gal activity was carried out according to standard procedures. Dissected TA muscles were fixed for 1 hour at 4° C. in 0.2% glutaraldehyde in PBS. Fixed muscles were washed three times in rinse solution (0.005% Nonidet P40 and 0.01% sodium deoxycholate in PBS). Muscles were stained overnight at 30° C. using a standard staining solution (5 mM potassium ferricyanide, 5 mM potassium ferrocyanide, 2 mM MgCl₂, 0.4% X-gal in PBS), rinsed twice in PBS and posffixed in 3.7% formaldehyde. Muscles were embedded in paraffin and then sectioned to observe LacZ expression at the cellular level. Sections were cut at 7-μm thickness and, after microscopic examination for the presence of β-gal positive myofibers, counterstained with hematoxylin and eosin. To perform immunostaining for human β2M, sections were deparaffinized and blocked by incubation for 30 minutes at room temperature with sheep anti-mouse Ig (Chemicon, Hofheim, Germany) diluted 1:50 in PBS. For light microscopy, endogen peroxidase was blocked with 0.5% H₂O₂ in methanol for 30 minutes. Sections were then incubated for 1 hour with a mouse anti-human (β2M monoclonal antibody (PharMingen, San Diego, Calif.) diluted 1:50 in PBS. This IgM antibody reacts specifically with human β2M (Liechty et al. in Nat Med (2000) 6, 1282-1286). Negative controls were sections from uninjected TA muscles incubated with primary antibodies, and sections from human SM-MPC injected-TA muscles incubated with normal mouse IgM isotype control instead of primary antibody.

For light microscopy, immunoreactivity was detected using the peroxidase-based EnVision™ System (Dako, Heverlee, Belgium). Sections were incubated for 30 minutes with horseradish peroxidase (HRP)-conjugated goat anti-mouse secondary antibody. A high sensitivity diaminobenzidine (DAB) chromogenic substrate system and Mayer's hematoxylin were used to visualize the immunoperoxidase and to counterstain nuclei, respectively. For examination by TEM, immunoreactivity was detected using silver enhanced pre-embedding colloidal-gold immunohistochemistry, using the following procedure. Sections were incubated in gold-conjugated goat anti-mouse secondary antibody (Aurion, Wageningen, The Netherlands) at a dilution of 1:15 in PBS containing Aurion BSA-C for 90 minutes, subsequently fixed in 2% glutaraldehyde in 0.05 M cacodylate buffer (pH 7.3) for 5 minutes, and finally silver enhanced (Aurion) for 16 minutes. After each step, sections were extensively washed in PBS. After immunolabelling, sections were postfixed in 2% osmium tetroxide for 1 hour, stained with 2% uranyl acetate in 10% acetone for 20 minutes, dehydrated through graded concentrations of acetone, and directly embedded on the plastic coverslip in Araldite™ according to the “Pop-Off technique”. Ultrathin sections (0.2 μm) were mounted on 0.7% formvar coated grids, stained with uranyl acetate and lead citrate for examination with a Philips EM 208 transmission electron microscope operated at 80 kV.

For fluorescent ISH, mouse centromeric probe was generated by PCR on mouse DNA using primers derived from the 120 bp consensus sequence within the mouse minor satellite DNA. Primers were: sense 5′-GGAAAATGATAAAAACCACACTG-3′ [SEQ ID NO: 1]; reverse 5′-TGTTTCTCATTGTAACTCATTGAT-3′ [SEQ ID NO: 2]. The human centromere 18 (CEN18) probe was generated by labeling BAC DNA (RPCI-373M8), containing centromere 18 sequences. Labeling of the human CEN18 DNA and the mouse centromeric PCR product with, respectively, fluoresceine-dUTP and lissamine-dUTP was performed using the BioNick™ Labeling System kit (Life Technologies). Frozen sections were treated 10 minutes with pepsin (10 mg pepsin in 100 ml 0.01 N HCl) at 37° C., washed with PBS, and fixed in 1% acid-free formaldehyde solution (PBS containing 50 mM MgCl₂, 1% acid-free formaldehyde). After washing with PBS, slides were dehydrated and air-dried. Chromosomes were denatured by incubating the slides at 72° C. in a 70% formamide/2×SSC solution, and dehydrated through ice-cold ethanol series. Probes were denatured in hybridization mixture (50% forrnamide, 2×SSC, 10% dextrane-sulfate) for 5 minutes at 75° C., and applied onto the slides, which were let hybridize overnight at 37° C. The following day, slides were washed 1 minute in 0.4×SSC/0.3% NP40 at 73° C., 1 minute in 2×SSC/0.1% NP40 at room temperature, and 5 minutes in 4T (4×SSC, 0.05% Tween 20; pH 7.0) at room temperature. Slides were dehydrated and mounted with antifade medium (Vectashield, Vector Laboratories, Burlingame, UK) containing DAPI to visualize nuclei. Analysis was performed with a Zeiss Axioskop2 using Cytovision software (Applied Imaging, Newcastle upon Tyne, UK).

Image acquisition and analysis: Digital images were acquired using SPOT camera and Spot software version 3.0.4 (Diagnostic Instruments, Sterling Heights, Mich.). Within the same experiment, the same color, and at the same magnification, fluorescent images were obtained using the same exposure settings. To ensure a perfect superimposition, brightfield, fluorescent red, and fluorescent green images were obtained separately, changing the light source and the filters but neither the position of the slide nor the focus. When needed, digital images were superimposed and treated for best rendering using Adobe® Photoshop® 6 (Adobe Systems Benelux BV, Amsterdam, The Netherlands).

Total RNA extraction and reverse transcription (RT)-PCR analysis: Total RNA was isolated using TRIzol reagent (Life Technologies) according to the manufacturer's instructions. After DNAse (Invitrogen) treatment, complementary DNA (cDNA) were obtained by RT of 2 μg (microgram) of total RNA (Thermoscript; Life Technologies) using oligo(dT)₂₀ as primer. Semiquantitative PCR was performed as described in De Bari et al. cited supra. Genomic DNA contamination was excluded by (a) primers spanning an intron, when possible, and (b) RT reactions without reverse transcriptase. Gene expression of human cells within muscle tissues was evaluated using primers specific for human cDNA. When mouse/human chimeric samples were equalized for the expression of human β-actin, control mouse muscle samples with no human cells were normalized to the mouse/human chimeric sample of the series with the highest mouse/human β-actin. In the time-point semiquantitative RT-PCR analysis of the human SM-MPC myogenic differentiation in vivo, PCR reactions were stopped at reaching plateau levels in at least one of the samples. The sequences of the primers and the expected sizes of the amplification products of the different examples of the present invention are listed in Table 1.

TABLE 1

genes and expected sizes of amplification products
obtained by amplification with sense and antisense
primers.

| | |
|---|---|
| mouse/human-β-actin (661 bp) | |
| sense | |
| 5′-TGACGGGGTCACCCACACTGTGCCCATCTA- | [SEQ ID NO: 6] |
| 3′ | |
| antisense | |
| 5′-CTAGAAGCATTTGCGGTGGACGATGGAGGG- | [SEQ ID NO: 7] |
| 3′ | |
| human-β-actin (662 bp) | |
| sense | |
| 5′-CCGACAGGATGCAGAAGGAG-3′ | [SEQ ID NO: 8] |
| antisense | |
| 5′-GGCACGAAGGCTCATCATTC-3′ | [SEQ ID NO: 9] |
| human-PCNA (548 bp) | |
| sense | |
| 5′-GGAGAACTTGGAAATGGAAAC-3′ | [SEQ ID NO: 10] |
| antisense | |
| 5′-CTGCATTTAGAGTCAAGACCC-3′ | [SEQ ID NO: 11] |
| human-myf5 (417 bp) | |
| sense | |
| 5′-TGAGAGAGCAGGTGGAGAACTAC-3′ | [SEQ ID NO: 12] |
| antisense | |
| 5′-GCCTTCTTCGTCCTGTGTATTAG-3′ | [SEQ ID NO: 13] |
| human-myogenin (565 bp) | |
| sense | |
| 5′-GCCACAGATGCCACTACTTC-3′ | [SEQ ID NO: 14] |
| antisense | |
| 5′-CAACTTCAGCACAGGAGACC-3′ | [SEQ ID NO: 15] |
| human-desmin (519 bp) | |
| sense | |
| 5′-CCTACTCTGCCCTCAACTTC-3′ | [SEQ ID NO: 16] |
| antisense | |
| 5′-AGTATCCCAACACCCTGCTC-3′ | [SEQ ID NO: 17] |
| human-dystrophin (566 bp) | |
| sense | |
| 5′-CAGTAGCCCCATCACATTTG-3′ | [SEQ ID NO: 18] |
| antisense | |
| 5′-ATAACGCAATGGACAAGTGG-3′ | [SEQ ID NO: 19] |
| human-SkM-actin (597 bp) | |
| sense | |
| 5′-CGTGGCTACTCCTTCGTGAC-3′ | [SEQ ID NO: 20] |
| antisense | |
| 5′-CCCATTGAGAAGATTCGTCG-3′ | [SEQ ID NO: 21] |
| human-MCK (721 bp) | |
| sense | |
| 5′-GGCACAATGACAACAAGAGC-3′ | [SEQ ID NO: 22] |
| antisense | |
| 5′-GAAAAGAAGAGGACCCTGCC-3′ | [SEQ ID NO: 23] |
| human-MyHCIIx/d (599 bp) | |
| sense | |
| 5′-ATAGGAACACCCAAGCCATC-3′ | [SEQ ID NO: 24] |
| antisense | |
| 5′-TTTGCGTAGACCCUGACAG-3′ | [SEQ ID NO: 25] |
| human-osteocalcin (362 bp) | |
| sense | |
| 5′-TCACACTCCTCGCCCTATTG-3′ | [SEQ ID NO: 26] |
| antisense | |
| 5′-GAAGAGGAAAGAAGGGTGCC-3′ | [SEQ ID NO: 27] |
| human-α1 (IX) collagen (363 bp) | |
| sense | |
| 5′-ACTGGGTTCTCTGGGTAGCC-3′ | [SEQ ID NO: 28] |
| antisense | |
| 5′-ATGTGCTGATCTGTCGGTGC-3′ | [SEQ ID NO: 29] |
| human-aP2 (290 bp) | |
| sense | |
| 5′-TATGAAAGAAGTAGGAGTGGGC-3′ | [SEQ ID NO: 30] |
| antisense | |
| 5′-CCACCACCAGTTTATCATCCTC-3′ | [SEQ ID NO: 31] |
| human-MGF (243 bp) | |
| sense | |
| 5′-TCTTCAGTTCGTGTGTGGAGAC-3′ | [SEQ ID NO: 32] |
| antisense | |
| 5′-TTGTTGGTAGATGGGGGCTG-3′ | [SEQ ID NO: 33] |
| murine-MGF (241 bp) | |
| sense | |
| 5′-TTCAGTTCGTGTGTGGACCG-3′ | [SEQ ID NO: 34] |
| antisense | |
| 5′-TTGTTTGTCGATAGGGACGG-3′ | [SEQ ID NO: 35] |
| humanFLK-1 (646 bp) | |
| sense | |
| 5′-TGTTGTTCTTTCCACCAGCAG-3′ | [SEQ ID NO: 36] |
| antisense | |
| 5′-ACGGTCTGGAAGGAACTCTC-3′ | [SEQ ID NO: 37] |
| human CDMP1 (595 bp) | |
| sense | |
| 5′ GCCCTGTTCCTGGTGTTTGG-3′ | [SEQ ID NO: 38] |
| antisense | |
| 5′ GCTGTGTAGATGCTCCTGCC-3′ | [SEQ ID NO: 39] |
| human MyoD (723 bp) | |
| sense | |
| 5′-ACGGCATGATGGACTACAGC-3′ | [SEQ ID NO: 40] |
| antisense | |
| 5′-CACCTGCTACATTTGGGACC-3′ | [SEQ ID NO: 41] |
| human c-MET (750 bp) | |
| sense | |
| 5′-CCAATGTCCTCTCGCTCCTG-3′ | [SEQ ID NO: 42] |
| antisense | |
| 5′-AGAAGGAGGCTGGTCGTGTG-3′ | [SEQ ID NO: 43] |
| human CD44 (661 bp) | |
| sense | |
| 5′-TTGGAGATGGATTCGTGGTC-3′ | [SEQ ID NO: 44] |
| antisense | |
| 5′-GGACTCTTGGACTCTTCTGG-3′ | [SEQ ID NO: 45] |
| human CD90 (366 bp) | |
| sense | |
| 5′-ATGAACCTGGCCATCAGCATCGC-3′ | [SEQ ID NO: 46] |
| antisense | |
| 5′-CTGTGACGTTCTGGGAGGAG-3′ | [SEQ ID NO: 47] |

Results

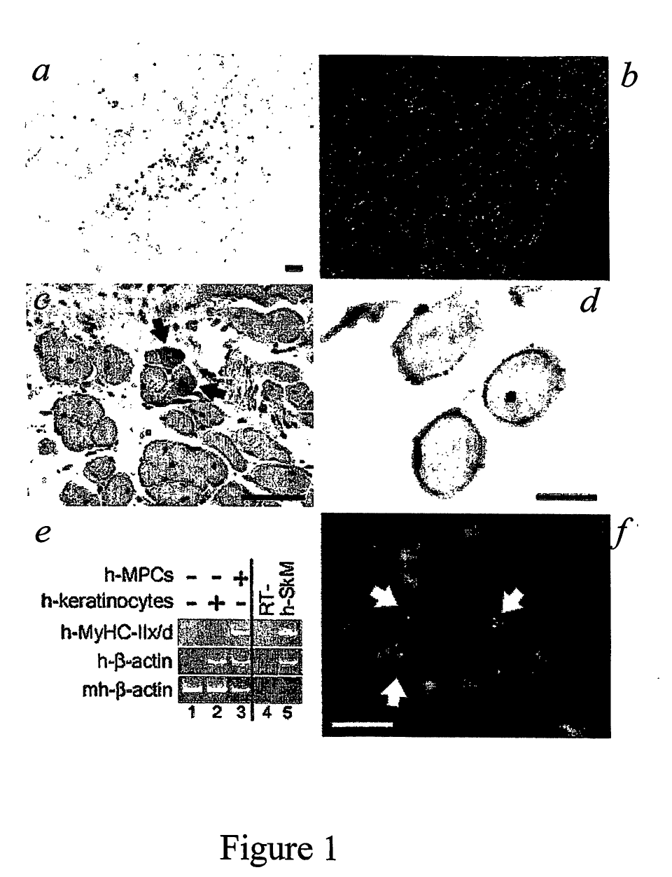

Skeletal muscle is a syncytial tissue. To localize the injected human cells, the nucleus was traced, which is the only cell structure that possibly preserves its individuality upon cell fusion, by using in situ hybridization (ISH) for human-specific ALU genomic repeats. Four weeks after implantation, a variable number of human nuclei were detected in 60% (109/180) of the stained longitudinal sections throughout the mouse TA muscle (FIG. 1a and b). The staining was specific, as sections from uninjected mouse TA muscles were all negative. The ALU positive nuclei were mostly located at the borders of the muscle fibers (FIGS. 1a and b). Using light microscopy, it is not possible to distinguish whether these nuclei were integrated into muscle fibers, or if they belonged to cells that were situated between the myofibers, but maintained their individuality. To address this issue, two strategies were followed. First, human SM-MPCs transduced with an adenovirus containing the LacZ gene encoding bacterial β-galactosidase (β-gal, beta-gal) were implanted into regenerating TA muscle. Mice were killed after 1 and 3 weeks. At 1 week, numerous β-gal positive mononuclear cells were detected in between the myofibers, but no β-gal positive myofibers were detected. At the 3-weeks time-point, some myofibers displayed a diffuse β-gal expression (arrows) especially in areas of regeneration, with fibers of heterogeneous size and central location of myonuclei (FIG. 1c), demonstrating the incorporation of at least 1 transduced human cell for each β-gal positive fiber. Counterstaining in FIG. 1c is performed with hematoxylin and eosin. Contralateral TA muscles injected with cells transduced with control adenovirus were negative. The second strategy consisted of staining sections of TA muscles for human β2-microglobulin (β2M, beta2M). After 4 weeks, in the TA muscle injected with human SM-MPCs, some myofibers expressed human β2M, with a pattern mostly limited to the sarcolemma (FIG. 1d). Muscle fibers with a cell membrane-associated staining are shown. Nuclei are counterstained with hematoxylin. Sections from uninjected TA muscles were all negative. The muscle fibers contributed by human cells were mostly located in regenerating areas, as suggested by the heterogeneous size of the myofibers and the central location of their myonuclei (Gillis cited supra).

It was also investigated whether the human cells implanted in the mouse TA muscles acquired the skeletal muscle phenotype, by performing RT-PCR with primers specific for human cDNA. Human myosin heavy chain type IIx/d (MyHC-IIx/d) were detected in the TA muscles four weeks after injection with human SM-MPCs (lane 1 FIG. 1e). Under similar experimental conditions, human MyHC-IIx/d was not detected in TA muscles injected with human keratinocytes, though human β-actin was detectable (FIG. 1e lane 3) For a tissue negative control for human gene expression, the CTX-treated muscle was injected with PBS (lane 1). Lane 4 is a, RT negative control of lane 3; lane 5 is human skeletal muscle as a positive control for human gene expression.

Cell nuclear fusion has been suggested as a possible explanation for stem cell plasticity (Terada et al. in Nature (2002) 416, 542-545; Ying et al. in Nature (2002) 416, 545-548.) The occurrence of fusion hybrids between donor human SM-MPC nuclei and recipient mouse muscle nuclei in vivo was investigated, which could explain the nuclear reprogramming underlying human SM-MPC myogenic differentiation. To do so, a genetic analysis was performed by double fluorescent ISH using a probe for human centromere 18 and a probe for mouse centromeric satellite DNA on longitudinal cryostat sections of regenerating TA muscles 4 weeks after human SM-MPC transplantation (FIG. 1f). Over 160 human nuclei counted in 2 sections, 154 (96%) were undoubtedly distinct from mouse nuclei. The remaining 6 human nuclei were located in areas where mouse and human cells were clustered, rendering thereby impossible to distinguish between overlapping or fused nuclei. Taken together, these findings provide evidence that implanted human SM-MPCs can efficiently fuse with host myofibers and contribute their genetic information to the mosaic myofibers generated. The detection of human MyHC-IIx/d shows that a number of human cells acquired the skeletal muscle phenotype.

### Example 2

### The in vivo Myogenic Potential is Independent of Donor Age or Cryopreservation, and is Conserved in Clonal Cells

Methods:

Immunofluorescence staining for human dystrophin, was done with the monoclonal antibody NCL-DYS3 (Novocastra Laboratories, Newcastle, UK), which does not stain mouse dystrophin (Huard et al. in Cell Transplant (1993) 2, 113-118). Unfixed frozen sections were air-dried and blocked for 1 hour at room temperature with sheep anti-mouse Ig diluted 1:50 with 1% Blocking Reagent in PBS supplemented with 0.2% Triton X-100. After rinsing in 1% Blocking Reagent in PBS, slides were incubated for 1 hour at room temperature with the mouse anti-dystrophin monoclonal antibody, diluted 1:20 in 1% Blocking Reagent in PBS. Slides were then extensively washed with PBS containing 0.2% Triton X-100, and incubated for 1 hour at room temperature with a Cy3-conjugated goat anti-mouse IgG antibody (Jackson ImmunoResearch Lab.), diluted 1:200 in 1% Blocking Reagent in PBS. After 3 washes in PBS-0.2% Triton X-100, slides were mounted in Mowiol containing DAPI for nuclear staining. For a positive control, sections from human skeletal muscle were used. Tissue negative controls were sections from mouse TA muscles as well as from PBS-injected mdx TA muscles incubated with the primary antibody. For the isotype control, sections from human MPC-injected TA muscles were incubated with normal mouse IgG instead of primary antibody.

Results:

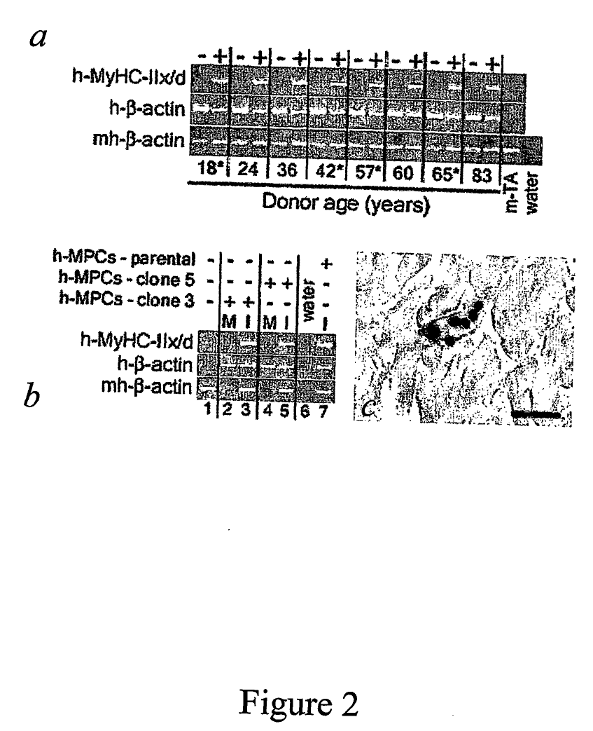

Reproducibility of the in vivo myogenic assay was tested with SM-MPCs from 8 adult human donors of various age. Asterisks in FIG. 2a indicate cells that had been frozen in liquid nitrogen. SM-MPCs before implantation (−) and TA muscles 4 weeks after SM-MPC implantation (+) were subjected to RT-PCR analysis for the expression of human MyHC-IIx/d. Human MyHC-IIx/d was not detected in culture expanded SM-MPCs in all experiments performed. In contrast, mouse TA muscles injected with SM-MPCs consistently expressed human MyHC-IIx/d, regardless of donor age, within the ranges examined, or cell storage in liquid nitrogen for up to 36 months (FIG. 2a). Mouse TA muscle was used to show the specificity of the primers for human cDNA.

The multilineage potential of human SM-MPCs is inherent to clonal cells in vitro as described in De Bari et al cited supra. To assess the potential of clonal SM-MPCs for skeletal muscle differentiation in vivo, 2 SM-MPC clones were tested (De Bari et al. cited supra). After 3 weeks, both TA muscles injected (I) with either clonal cell populations expressed human MyHC-IIx/d (FIG. 2b. lanes 3 and 5), with levels comparable to the TA muscle implanted with the parental cell population (lane 7). cDNA templates were equalized for the expression of human β-actin. Human MyHC-IIx/d was not expressed at detectable levels by SM-MPC clones in monolayer (M) before implantation (lanes 2 and 4). Lane 1, mouse CTX-treated TA muscle; lane 6, Milli-Q water negative control.

Other genes of the mature skeletal muscle phenotype, such as MCK and dystrophin, were also expressed. Chains of human nuclei were detected by ISH for ALU genomic repeats performed on paraffin sections in the center of muscle fibers in areas containing fibers of variable diameters indicating a contribution to the compartment of myonuclei (FIG. 2c). A muscle fiber with a chain of human nuclei, stained black, is shown (counterstaining with eosin). These data provide evidence that the in vivo myogenic potential is inherent to individual cell clones of multipotent SM-MPCs.

### Example 3

### SM-MPC Differentiation Recapitulates Embryonic Myogenesis

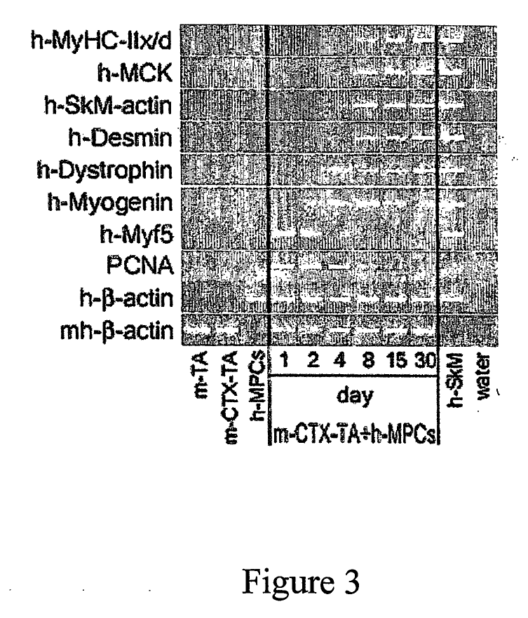

This example shows that the mature skeletal muscle phenotype of the human cells was acquired through a cascade of molecular events reminiscent of embryonic myogenesis. This in vivo assay can be considered a chimeric experiment where human cells have been injected into a mouse host, thereby offering the possibility to monitor selectively the phenotype of the injected (human) cells within the entire TA muscle. A time-point semiquantitative RT-PCR gene expression analysis of the human SM-MPC differentiation was carried out on muscle samples obtained at several time points after injection of SM-MPC, by using primers specific for human cDNA. TA muscle samples containing human cells were normalized for the expression of human β-actin. Since PCR reactions were stopped at reaching plateau levels in at least one of the samples, the experiment is informative about temporal dynamics of expression for each gene separately. An early peak of human Myf5 and PCNA was observed already 24 hours after cell implantation, indicating commitment to skeletal myogenesis coupled with an early wave of cell proliferation. With the decline of Myf5, the expression of human myogenin increased, peaking at 8 days, and then decreased, while the mature markers such as MyHC, mouse creatine kinase (MCK), and dystrophin progressively reached plateau levels (FIG. 3). PCR reactions were stopped at reaching plateau levels in at least one of the samples. For each time-point, a PBS-injected regenerating (CTX-treated) mouse TA muscle was included for a negative control. m-TA=uninjected mouse TA muscle; m-CTX-TA=mouse CTX-treated TA muscle; h-MPCs=human muscle progenitor cells; h-SkM=human skeletal muscle; MCK=muscle creatine kinase; PCNA=proliferating cell nuclear antigen.

These data show that contribution of SM-MPCs to skeletal muscle regeneration, in this model, is a multistep process that appears to recapitulate embryonic muscle formation.

Expression by human SM-MPCs of muscle-specific genes may be the consequence of reprogramming of human nuclei secondary to fusion into host muscle fibers (Blau et al. in Science (1985) 230, 758-766). Firstly, the ability in vitro, under specific conditions, of some human SM-MPCs to form myotubes expressing MyHC (De Bari et al. cited supra) would already suggest that muscle cytoplasm is not required for myogenic differentiation and muscle gene expression. In addition, it is shown that an early-immediate peak of Myf5 arises with a steep decrease within 48 hours, followed by a peak of myogenin after 8 days. Under our experimental conditions, we did not observe muscle fibers contributed by human cells before 7 days. Although sporadic early fusion with host muscle fibers can not be excluded, it is documented that immediate-early skeletal muscle-specific differentiation events are happening and even extinguishing before fusion with myofibers could be documented.

Since Myf5 was not detectable in the original SM-MPC population, it is likely that the high expression of human Myf5 already 24 hours after SM-MPC implantation was due to gene induction/upregulation instead of enrichment of Myf5 expressing cells. During embryonic development, Myf5 is necessary to restrict undifferentiated cells to myogenesis (Tajbakhsh et al. in Nature (1996) 384, 266-270). Likewise, during human MPC differentiation Myf5 may determine cell specification and commitment to myogenesis. While Myf5 was progressively disappearing, the human myogenic differentiation program was proceeding with the second muscle-specific key molecular event, the peak of myogenin, followed by plateau levels of mature muscle markers. Taken together, our findings suggest that human SM-MPCs undergo a long and multistep differentiation process, which comprises cell division, commitment to the myogenic lineage, and eventual terminal maturation and fusion. The kinetics of muscle differentiation of human SM-MPCs appear to be very similar to those of BM-derived cells, as it was reported that myoblasts injected into regenerating TA muscle fused into muscle fibers within 5 days, whereas BM-derived cells required at least 2 weeks for integration into muscle fibers (Ferrari et al cited supra).

### Example 4

### Contribution to Functional Satellite Cells

Methods:

Myoblast isolation and transplantation: Primary skeletal myoblasts were isolated as described in Salvatori et al. (in Hum Gene Ther (1993) 4, 713-723), with a few modifications. TA muscles of 6 nude mice (14 months of age), which had been transplanted with human SM-MPC 6 months earlier, were dissected free from skin, and minced into pieces of about 1 mm³. Cells were released by digestion in 10 mg/ml dispase (Sigma) at 37° C. for 2 hours, and in 0.2% collagenase (Life Technologies) at 37° C. for 1 hour, and filtered through a 70 μm nylon mesh (Life Technologies). Dissociated single cells were washed twice in PBS, and were then plated on plastic Petri dishes and maintained for 2 hours at 37° C. in growth medium to allow attachment of fibroblasts. Nonadherent cells were collected and plated on gelatin-coated culture plates in DMEM supplemented with 20% FBS and antibiotics. Differentiation to myotubes was induced by starvation, exposing confluent myoblast culture to DMEM containing 2% horse serum for 48 hours. At 70% confluence, when no myotubes were observed, myoblasts were trypsin-released, washed with PBS, and implanted into regenerating TA muscles. The expanded myoblast population contained both mouse and human cells. To avoid injecting too few human cells, each of the 2 injections was made with 3×10⁶ viable cells.

To perform double immunofluorescence staining for laminin and human β2M, frozen sections were air-dried, fixed in buffered 4% paraformaldehyde for 10 minutes at room temperature, and washed extensively with PBS. Slides were incubated twice in 50 mM ammonium chloride in PBS for 10 minutes to quench autofluorescence, washed in PBS, and blocked for 1 hour at room temperature with sheep anti-mouse Ig diluted 1:50 with 1% Blocking Reagent (Roche Molecular Biochemicals, Brussels, Belgium) in PBS supplemented with 0.2% (v/v) Triton X-100 (Bio-Rad, Nazareth Eke, Belgium). After rinsing twice in 1% Blocking Reagent in PBS to remove the excess of sheep anti-mouse Ig, slides were incubated overnight at 4° C. with 1% Blocking Reagent in PBS containing 10 μg/ml rabbit anti-laminin polyclonal antibody (InnoGenex, San Ramon, Calif.) and mouse anti-human β2M monoclonal antibody diluted 1:50. Slides were then extensively washed with PBS containing 0.2% Triton X-100, and incubated for 1 hour at room temperature with Cy3-conjugated goat anti-rabbit IgG (H+L) antibody (Jackson ImmunoResearch, West Grove, Pa.) and Cy2-conjugated goat anti-mouse IgG (H+L) antibody (Kirkegaard & Perry Laboratories, Gaithesburg, Md.), both diluted 1:200 in 1% Blocking Reagent in PBS. Since light chains are common to IgG and IgM, the secondary antibody reacts with both Ig isotypes. After 3 washes in PBS-0.2% Triton X-100, nuclei were counterstained with 4′,6-diamidino-2-phenylindole (DAPI; ICN, Asse-Relegem, Belgium) and mounted with Mowiol (Calbiochem-Merck Belgolabo, Overijse, Belgium). Negative controls were sections from uninjected TA muscles incubated with primary antibodies and sections from human SM-MPC-injected TA muscles incubated with normal rabbit serum and normal mouse IgM isotype control instead of primary antibodies.

Results

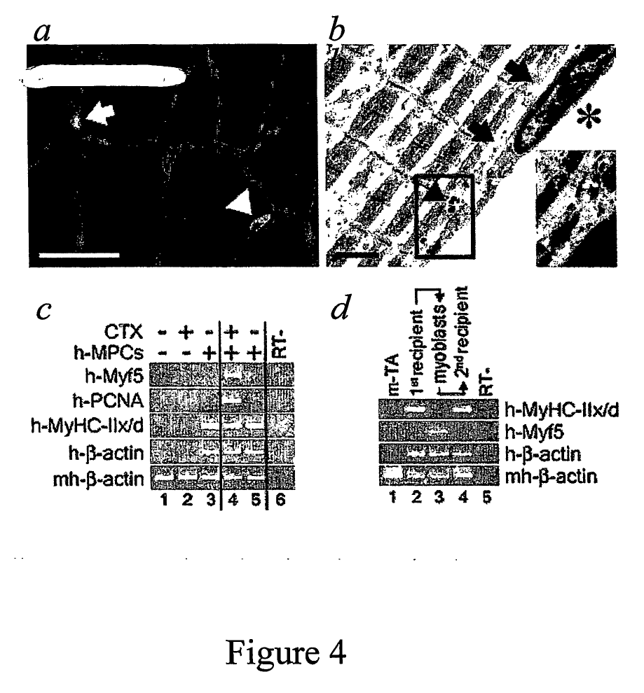

The potential of skeletal muscle to regenerate after injury relies on the persistence of a reserve population of undifferentiated mononuclear cells, termed satellite cells, which lie beneath the basal lamina, applied to the sarcolemma of myofibers. To test the possibility that implanted human cells can survive as satellite cells, a double immunostaining was performed for laminin, identifying the basal lamina, and human β2M, identifying the human cells, on sections of TA muscles 6 months after human SM-MPC transplantation. A number of human β2M-positive mononuclear cells (arrowhead, white) were detected residing between basal lamina and muscle fibers (grey) (FIG. 4a). The staining for human β2M was confined to the mononuclear cells and did not extend to the sarcolemma of the adjacent myofibers, indicating that the human cells had not fused with mouse muscle fibers.

Given the absence of specific markers, the most reliable way to identify satellite cells remains electron microscopy (Grounds et al. in J Histochem Cytochem (2002) 50, 589-610). Specific ultrastructural criteria are as follows: a plasma membrane separating the satellite cell from its adjacent muscle fiber, an overlying basal lamina continuous with the satellite cell and associated fiber, and the heterochromatic appearance of the nucleus (Bischoff cited supra). Transmission electron microscopy (TEM) was used on sections of TA muscles of nude mice transplanted 6 months earlier with human SM-MPCs, after staining with the antibody specific for human β2M. Some mononuclear cells with a plasma membrane-associated staining for human β2M fulfilled the ultrastructural criteria of satellite cells (FIG. 4b). The high magnification (scale bar: 100 nm) of a satellite cell revealed a plasma membrane (arrows) positive for human β2M (arrowhead), separating the satellite cell from its adjacent myofiber, the continuous basal lamina (asterisk) surrounding the satellite cell and myofiber, and the heterochromatic appearance of the nucleus. Inset shows an inverted, high-magnified view of the silver grains of the staining for human β2M.

These results demonstrate that a subpopulation of the implanted human SM-MPCs can survive for a long period of time as mononucleated cells, with the typical spatial location and morphology of satellite cells.

In mature skeletal muscle, satellite cells are normally quiescent and are activated in response to environmental cues, such as injury, to mediate postnatal muscle regeneration. Functional satellite cells respond to muscle injury with coordinated proliferation and expression of activation markers, such as Myf5 (Cornelison & Wold in Dev Biol (1997) 191, 270-283). To test whether human SM-MPCs contributed long-term persisting cells that would function in vivo as satellite cells, right TA muscles of 3 mice were injured by CTX injection that, 6 months earlier, had received human SM-MPCs bilaterally into regenerating TA muscles. Contralateral TA muscles received PBS. Animals were killed twenty-four hours later. Semiquantitative RT-PCR revealed strong upregulation of human PCNA and human Myf5 in the CTX-injured TA muscles (lane 4 FIG. 4c) as compared to the CTX-untreated contralateral muscles (lane 5 FIG. 4c) indicating that the human cells transplanted 6 months earlier were capable of activation upon injury, with a satellite cell-like response. Controls were: mouse TA muscle (lane 1); mouse TA muscle 24 hours after CTX treatment (lane 2); mouse CTX-treated TA muscle implanted with human SM-MPC and harvested after 6 months as external control (lane 3); RT negative control of lane 4 (lane 6).

Satellite cells are known to be able to form myotubes under low serum conditions in vitro, and to contribute to muscle repair when injected into a regenerating muscle in vivo (Seale & Rudnicki in Dev Biol (2000) 218, 115-124). To investigate whether the human SM-MPCs shared the same properties after they had contributed to the satellite cell compartment in vivo, cultures of satellite cell-derived primary myoblasts from mouse TA muscles were established that had been injected with human SM-MPCs 6 months earlier (first recipients). During in vitro expansion, human cell nuclei remained distinct from mouse cell nuclei, with no apparent fusion as determined by double genomic ISH. Proliferating primary myoblasts express specific markers such as Myf5, but not terminal differentiation markers such as MyHC (Cornelison & Wold in Dev Biol (1997) 191, 270-283; Smith et al. in J Cell Physiol (1994) 159, 379-385). Analogously, in cultures of first recipient satellite cell-derived primary myoblasts human Myf5 was detected, but not human MyHC-IIx/d (FIG. 4d). d, Human mononuclear cells recovered from first recipient mice retain in vivo myogenic activity when transplanted into a second recipient. Six months after implantation of human SM-MPC into regenerating TA muscles (Lane 1, control mouse TA muscle; lane 2, TA muscle from first mouse recipient; lane 3, first recipient satellite cell-derived primary myoblasts; lane 4, TA muscle from second mouse recipient; lane 5, RT negative control of lane 4). Under low serum conditions, cells underwent terminal differentiation and formed multinucleated myotubes that expressed human MyHC. This property was not shared by SM-MPCs before implantation in the first recipients. At about 70% confluence, when no myotubes were observed, the first recipient satellite cell-derived primary myoblasts were implanted into regenerating TA muscles of second recipient mice. One month later, human MyHC-IIx/d mRNA (FIG. 4d), was detected and human nuclei in 10% (8/80) of the stained sections were found throughout the muscle, indicating that recovered human cells contributed to muscle regeneration in second recipients. On the basis of these results, it is concluded that upon implantation, human SM-MPCs can give rise to functional satellite cells.

### Example 5

### Differentiation is Sensitive and Specific to Environmental Cues

Methods: CTX induced damage of TA muscle was performed as described in example 1. For systemic injections, 5×10⁶ viable SM-MPCs in 250 μl DMEM were slowly infused into the bloodstream of a tail vein 24 hours after the CTX induced damage.

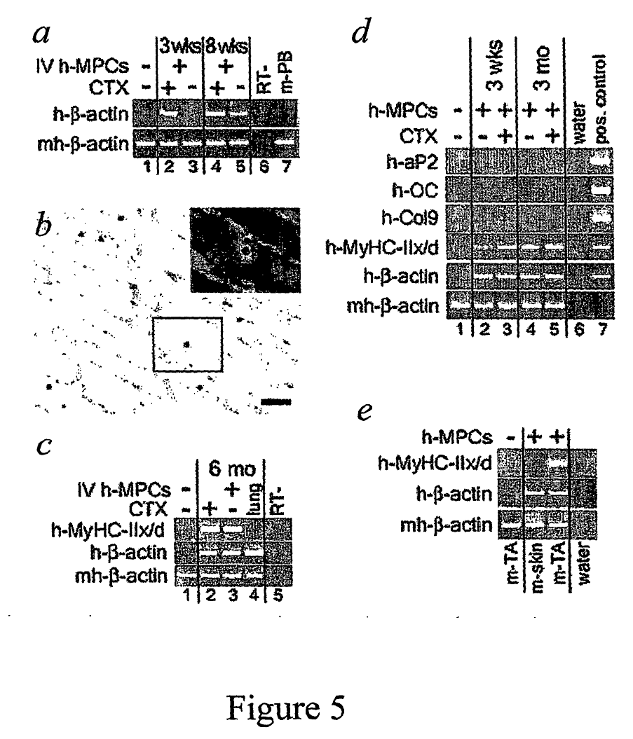

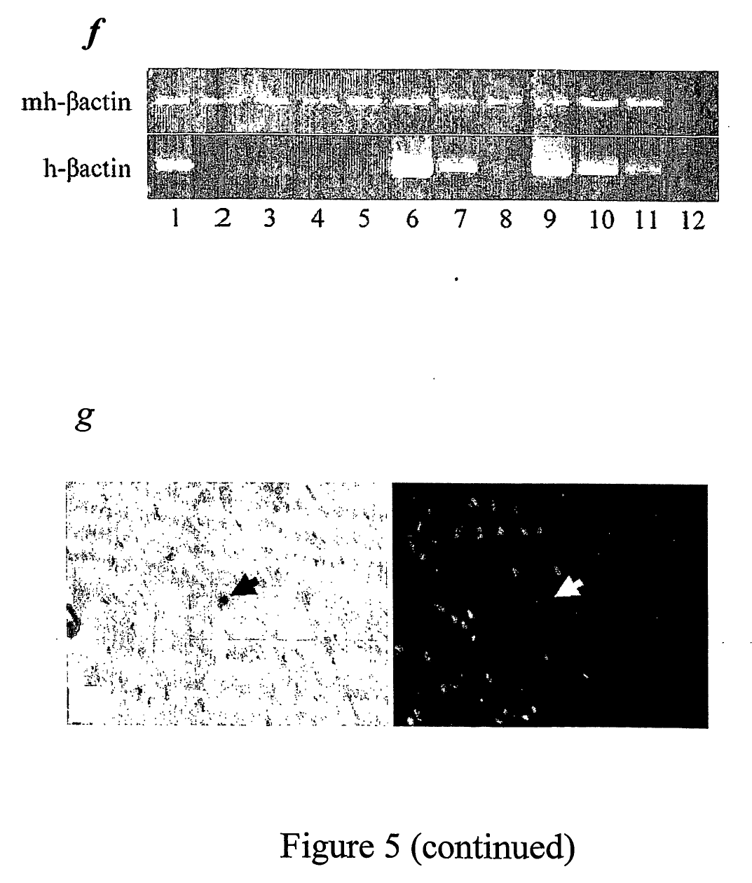

Results: Systemic delivery of cells to target tissue and organ systems is a potential treatment modality in regenerative medicine. It was tested whether human SM-MPCs preferentially homed to damaged skeletal muscle and contribute to muscle regeneration when delivered systemically. Twenty-four hours after the injection of CTX into the right TA muscles, 5×10⁶ human SM-MPCs were administered in the tail vein of 6 mice. Two animals were killed for each time-point examined. Using a 40-cycle PCR, at 3 weeks human β-actin was detected only in the CTX-injured TA muscles (lanes 2 and 3 FIG. 5a), while at 8 weeks it was expressed in both CTX-treated and -untreated TA muscles (lanes 4 and 5 FIG. 5a) (Lane 1: mouse TA muscle, lane 6: RT negative control of lane 2). Human β-actin was not detected in the peripheral blood in either case (lane 7 FIG. 5a) indicating that, if human cells were circulating in the bloodstream, their number was too low for detection. At the time points of 3 and 8 weeks, in the TA muscles human MyHC-IIx/d was not detected by RT-PCR. Nevertheless, the location of a certain number of human nuclei at the center of myofibers after 3 weeks was indicative of some contribution to muscle regeneration (FIG. 5b).

After 6 months, in both CTX-treated and -untreated TA muscles human β-actin was detected after 33 PCR cycles, and human MyHC-IIx/d after 40 cycles (FIG. 5c). At this time-point, human nuclei were mostly located at the periphery of myofibers, suggesting full maturation of the muscle fibers contributed by human SM-MPCs. Human cells were also found in other mouse tissues and organs, such as lungs (lane 4 FIG. 5c) and heart (lane 7 FIG. 5f and FIG. 5g. The expression of human MyHC-IIx/d and MCK, markers of the mature skeletal muscle phenotype, in those non-muscle tissues and organs of the injected animals, which contained human cells as determined by RT-PCR for human β-actin and/or ISH for human ALU genomic repeats, at all time-points examined. In particular, at the 6 months time-point the number of human cells in the lungs was at least as high as in TA muscles, as evaluated by β-actin expression levels, yet the skeletal muscle markers were undetectable. Taken together, these results indicate that human SM-MPCs can be delivered systemically to the target tissue, with early preferential but not exclusive homing to the damaged skeletal muscle, and long-term contribution to skeletal muscle regeneration. The expression of human MyHC-IIx/d was specific to skeletal muscle, with no apparent heterotopic muscle formation, suggesting a context-sensitive differentiation response of the human SM-MPCs.

Myogenesis is one of the mesenchymal differentiation pathways that can be undertaken by human SM-MPCs in vitro (De Bari et al cited supra) and in vivo as shown in the present invention. To rule out heterotopic tissue formation, and to investigate whether muscle damage was required for the recruitment into the myogenic differentiation program when SM-MPCs were locally implanted, the expression was analyzed of selected human marker genes of the mature mesenchymal lineages in mouse TA muscles, damaged or not with CTX and injected with human SM-MPCs. After 3 weeks, the CTX-treated TA muscles of 3 independent animals expressed human MyHC-IIx/d (FIG. 5d lane 3) at higher levels than the contralateral CTX-untreated muscles (lane 2). Consistent with this finding, the number of myofibers with centrally located human nuclei was higher in the CTX-treated TA muscles than the contralateral CTX-untreated muscles. After 3 months, both CTX-treated and -untreated TA muscles displayed expression levels comparable to the CTX-treated muscles at the 3 weeks time-point lane 4 and 5) After 45 PCR cycles, neither the CTX-treated nor the CTX-untreated TA muscles expressed genes of the mature non-muscle mesenchymal lineages examined (aP2-fatty acid-binding protein aP2-for adipose tissue, osteocalcin—OC—for bone, and type IX collagen—Col9—for cartilage). This experiment was performed in 3 independent mice, with similar results. Negative controls were uninjected mouse TA muscle (lane 1) and Milli-Q water (lane 6). Positive controls (lane 7) were human skeletal muscle for MyHC-IIx/d, human primary articular chondrocytes for collagen type IX, human trabecular osteoblasts for osteocalcin, human fat tissue for aP2.

Results comparable to CTX-untreated muscles were observed upon human SM-MPC transplantation into TA muscles of age-matched CTX-untreated mice, thereby ruling out significant contralateral TA muscle injury by CTX possibly circulating in the bloodstream in mice with unilateral TA muscle injection of the snake venom. These results suggest that the local muscle injury induced by CTX would accelerate the commitment of human SM-MPCs to the skeletal muscle differentiation program, yet is not required. Neither the CTX-treated nor the CTX-untreated TA muscles expressed genes of the mature non-muscle mesenchymal lineages examined (FIG. 5d), suggesting that differentiation of the human SM-MPCs is sensitive and specific to environmental cues.

Subcutaneous implantation of primary myoblasts can generate ectopic skeletal muscle (12). To rule out ectopic skeletal muscle formation and to assess tumorigenicity, human SM-MPCs were injected either subcutaneously into the back or intramuscularly into TA muscles of 4 nude mice. After 12 weeks, human β-actin was retrieved in both sites of cell implantation. TA muscles (FIG. 5e, lane 3), but not skin (lane 2), expressed human MyHC-IIx/d. Neither ectopic muscle nor tumor formation was observed subcutaneously, as determined by macroscopic and histological examination. No adverse effect(s), such as tumor development, after injection in nude mice of human SM-MPCs even at high doses (2×10⁷ cells) was encountered, regardless of the site and the way of administration. As a positive tumor cell control, 6 mice received human 293 cells both subcutaneously and intramuscularly (0.5×10⁶ cells/site). All animals inoculated with 293 cells developed large tumors (1-2 cm in lo diameter) at the injected sites within 2 to 3 weeks.

Systemically delivered MS-MPC were not only encountered in skeletal muscle, but also in cardiac muscle thus indicating that systemic application finds damaged muscle cells and repairs these, e.g. as would occur after myocardail infarction. We conclude that MS-MPC are selectively attracted by damaged muscle. Apart from the CTX induced TA muscle, the naturally occurring damage in muscle or the induced damage due to spreading of the CTX to other tissues is sufficient to attract precursor cells to both skeletal and cardiac muscle.

### Example 6

### Restoration of Mouse MGF in mdx Dystrophic Mice

Methods:

Dystrophin deficient mdx mice (C57BL/10ScSn DMD^{mdx}/J) were purchased from The Jackson Laboratory (Bar Harbor, Me., USA). Two-month-old mice were used for all experiments. Transplantation was performed by single-point injection of 1×10⁶ viable human SM-MPCs suspended in 25 μl PBS into the right TA muscle, while the left TA muscle served as internal control receiving PBS with no cells. TA muscles were not preirradiated or injured with a myonecrotic agent before transplantation. Recipient mice were immunosuppressed with FK506 (Fujisawa Pharmaceutical Co. Ltd., Osaka, Japan) administered intraperitoneally at the dose of 2.5 mg/kg per day (Kinoshita et al. in Muscle Nerve (1994) 17, 1407-1415) from the day of transplantation till the animals were killed 1 month after transplantation.

DNA injection and electric-pulse delivery. pCMV-full length human dystrophin plasmid pTG11025 (Braun et al. in Gene Ther (2000) 7, 1447-1457) was a kind gift from S. Braun (Transgene, Strasbourg, France). Animals were anesthetized during the whole procedure. The skin above TA muscles was shaved before injection. Fifty μg of pTG11025 in 50 μl of 0.9% NaCl were injected percutaneously into the right TA muscle of 6 mdx mice in 5 different sites (10 μl per site). Sham control injections were done with pCMV-LacZ. Immediately after naked DNA administration, transcutaneous electric pulses were applied to 3 mice (out of the 6 mdx mice injected) through two stainless steel plate electrodes placed on either side of the hindlimb as described in (Mir et al. in Proc Natl Acad Sci USA (1999) 96, 4262-4267). The animals were immunosuppressed with FK506 as described above, and killed 1 month after plasmid DNA injection.

Quantitative (TaqMan) PCR was carried out using Prism 7700 sequence detection system according to manufacturer's protocols (Applied Biosystems, Lennik, Belgium). PCR for mouse MGF was performed with SYBR green. Data were normalized to β-actin mRNA measured with the following primers: 5′-CTGGCACCCAGCACMTG-3′ [SEQ ID NO: 3], 5′-AGCGAGGCCAGGATGGA-3′ [SEQ ID NO 4], and TaqMan probe 5′-JOE-CCGCCGATCCACACGGAGTACTTG-TAMRA-3 [SEQ ID NO 5]′ (Applied Biosystems); expected size 89 bp. TaqMan PCR products were gel electrophoresed to ensure the presence of a single amplification product of the right size.

Results:

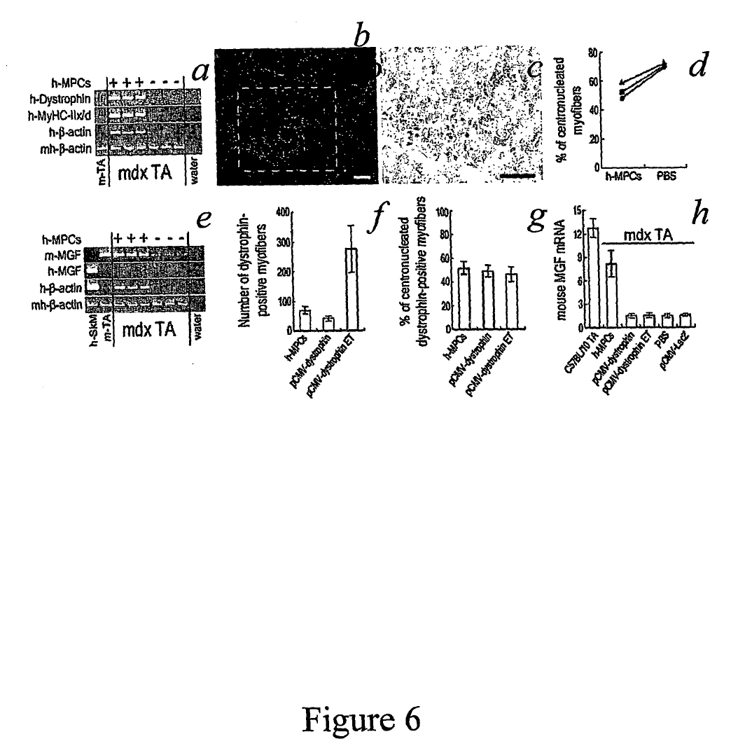

To explore whether human SM-MPCs can be employed to correct a genetic muscle disorder, the mdx mouse was adopted, a genetic and biochemical model of the human DMD. Skeletal muscles of the mdx mouse do not produce dystrophin protein due to a nonsense point mutation in exon 23 of the gene (Braun et al. in Gene Ther (2000) 7, 1447-1457). To investigate whether the human SM-MPCs were capable of myogenic differentiation in dystrophic muscles, human SM-MPCs were transplanted into the right TA muscles of three 2-month-old mdx mice. The left TA muscles were injected with vehicle solution, as internal controls. To limit rejection of the xenogeneic human cells, mice were immunosuppressed by intraperitoneal injection of FK506 (Kinoshita et al. in Muscle Nerve (1994) 17, 1407-1415). After 4 weeks, mdx TA muscles injected with human SM-MPCs (+) expressed human dystrophin and MyHC-IIx/d, while the contralateral PBS-injected TA muscles (−) did not (FIG. 6a).

To evaluate human dystrophin protein production and topography, a staining was performed using a monoclonal antibody that does not react with mouse dystrophin (Huard et al. in Cell Transplant (1993) 2, 113-118.). Therefore, putative revertant fibers expressing dystrophin protein by virtue of exon skipping (Lu et al. in J Cell Biol (2000) 148, 985-996) are not detected (Braun S et al. cited supra). Foci of myofibers expressing human dystrophin were observed in mdx TA muscles injected with human SM-MPCs; dystrophin immunoreactivity was located properly at the periphery of muscle fibers (FIG. 6b). Contralateral PBS-injected muscles were negative. Human nuclei were detected in a parallel, non-consecutive section in an area corresponding to the location of dystrophin-positive myofibers (FIG. 6c). These data indicate that human SM-MPCs retain the capacity of myogenic differentiation in dystrophic muscles, contributing to restoration of dystrophin.

To further examine the effect of human SM-MPC transplantation on the histology of the mdx muscle, the number of dystrophin positive myofibers that were centronucleated were investigated, compared to the contralateral PBS-injected TA muscle fibers counted after hematoxylin-eosin staining. The percentage of centronucleated myofibers in the human SM-MPC-injected muscles was significantly lower than the contralateral PBS-injected muscles (SM-MPCs, 53.1% vs. PBS, 71.0%; P<0.05) (FIG. 6d). These results show that human SM-MPC transplantation can restore, at least in part, the histology of dystrophic muscle for up to 4 weeks after transplantation.

Whether any function of mdx muscles could be rescued was evaluated by measuring the expression of mouse mechano-growth factor (MGF) by mdx muscles upon human SM-MPC transplantation. MGF is a splice variant of IGF-1 expressed by skeletal myofibers and upregulated in response to overload (Yang et al.cited supra). MGF appears to play a role in local muscle repair, maintenance, and remodeling (Yang et al. cited supra). It was reported that mRNA for MGF is not detectable in dystrophic mdx muscles by RT-PCR, even when subjected to stretch and stretch combined with electrical stimulation (Goldspink et al. in (1996) J. Physiol. 496P, 10) ). Therefore, the rescue of normal mouse MGF can be considered a measure of functional restoration of the mdx muscle. Compared to uninjected contralateral TA muscles, a dramatic and reproducible upregulation of mouse-specific MGF mRNA was observed 4 weeks after human SM-MPC implantation into mdx muscles, with expression levels comparable to the normal TA muscles from C57BL/10 mice, while human MGF was not detectable (FIG. 6d). Sequencing of the PCR product confirmed the specificity of the primers for mouse MGF.

To investigate whether dystrophin restoration is sufficient to rescue mouse MGF, plasmid DNA was injected containing full-length human dystrophin [pCMV-dystrophin, pTG11025 in (Braun et al. in Gene Ther (2000) 7, 1447-1457). into TA muscles of 3 immunosuppressed 2-month-old mdx mice. To increase the efficiency of in vivo gene transfer, plasmid DNA injection was followed by application of electric pulses in additional 3 age-matched mdx mice. After 4 weeks, proper sarcolemmal expression of human dystrophin was detected by immunostaining in transverse sections from pCMV-dystrophin-injected mdx TA muscles.

The maximal number of human dystrophin-positive myofibers per transverse section was 69.3 in TA muscles injected with SM-MPCs, 42.0 in TA muscles injected with pCMV-dystrophin, and 275 when electrotransfer (ET) was applied (FIG. 6f). There was no significant difference in the percentage of centronucleated dystrophin-positive myofibers (SM-MPCs, 53.1%, pCMV-dystrophin, 50.4%, pCMV-dystrophin ET, 47.3) (FIG. 6g). However, the expression levels of mouse MGF, as determined by quantitative RT-PCR, remained low in all pCMV-dystrophin-injected muscles, analogous to the PBS-injected or pCMV-LacZ-injected mdx muscles (FIG. 6h).

The level of mouse MGF expression induced after human MS-MPC is dramatically increased and reaches about 60 percent of the levels in healthy mice muscle. On longitudinal sections, dystrophin immunostaining was segmental in both SM-MPC-injected and pCMV-dystrophin-injected mdx TA muscles, extending over a stretch of approximately 100 to 700 μm, which reflects the dystrophin nuclear domain of previous studies (Gussoni et al. in Nat Med (1997) 3, 970-977; Kinoshita I et al. in Muscle Nerve (1998) 21, 91-103; Vilquin et al. in Gene Ther (2001) 8, 1097-1107).

### Example 7

### Molecular Characterisation of SM-MPCs

Populations of expanded cell from different tissues such as, articular cartilage, periosteum, stromal marrow and synovial membrane were inspected by light microscopy. It was an unexpected and surprising finding that expanded cell populations derived from synovial membrane contain more than 50% cells with a myofibroblast phenotype. Further, the inventors noticed a degree of correlation between the presence of myofibroblast like cells and the in vivo myogenic potential of a cell population. Thus, depending on the tissue source, isolation and expansion procedure the abundance of certain population of stem cell derived committed progenitor cells will be promoted. Also the synovial membrane derived cell population displays an abundant expression of the early myogenic marker c-MET. This makes synovial tissue a preferred source of MPC, over other parts of the joint or other tissues wherein prolonged expansions and more exhaustive selections with markers will be needed in order to obtain a cell population substantially enriched with MPC charisteristics.

Double immune fluorescence staining for c-Met and cdmp1 of human SM-MPC in monolayer after 8 passages shows that about 80% of the cell population is c-Met⁺, while only 5% are CDMP1⁺. Only a minor subpopulation of c-Met-positive cells (about 10%) co-express CDMP1. Thus, SM-MPC of the present invention can be characterized as a c-Met⁺/CDMP1⁻ cell population.

Table 2 presents an overview of positive and negative markers of the SM MPC as detected by RT PCR and is a compilation of markers described in De Bani et al (cited supra) and of markers identified in the present invention.

TABLE 2
Molecular markers of SM-MPCs as determined by RT PCR.

| | |
|---|---|
| Positive | Negative |

matrix molecules

| | |
|---|---|
| Aggrecan, Link protein, a1(I) | a1(II) collagen, a1(IX) collagen, |
| collagen, Lumican, Versican, | a1(X) collagen |
| Fibromodulin, Biglycan, Decorin | |

adhesion molecules

| | |
|---|---|
| b1 integrin, b5 integrin, av integrin, | PECAM-1 |
| CD44, VCAM-1 | |

Hematopoietic/endothelial markers
ligands

| | |
|---|---|
| BMP-2, BMP-4, BMP-6, TGFb1, | BMP-3, BMP-5, BMP-7, GDF- |
| TGFb2, TGFb3, Wnt5a | 6/CDMP-2, IHH |

Receptors

| | |
|---|---|
| ALK-1, ALK-2, ALK-3, ALK-4, ALK- | FGFR-3 |
| 5, ALK-6, BMPRII, FGFR-1, | |
| FGFR-2, Patched, Smoothened, | |
| CD90 | |

Transcription factors

| | |
|---|---|
| Sox9, Cbfa1 | Pax3 |

others

| | |
|---|---|
| Smad-1, Smad-2, Smad-5, Smad- | Osteopontin, Osteocalcin, |
| 7, Osteonectin, LBK-AP, Vimentin, | Myogenin, SkM-MHC, aP2, |
| a-actin, c-Met | PPARg2 |

* detectable at least in isolated cells and expanded cells at P0 and P3 and by FACS analysis of P8 cells.

The isolation procedure followed in the present invention for the isolation of SM MPCs excludes contamination with muscle tissue and muscle derived precursor cells. The isolation also excludes possible contamination by neural stem cells, liver cells, or dermal fibroblasts which are documented to have skeletal myogenic differentiation in vivo (Grounds cited supra). The SM-MPCs of the present invention also differ in their molecular characteristics with a number of muscle derived cell type as shown in table 3.

TABLE 3
molecular markers of SM-MPCs and muscle
derived myogenic precursors.

| SM MPC | mc13 | MDSC | ISSM **** | EDA SKP |
|---|---|---|---|---|
| (1) | (2) | (3) | (4) | (5) |

early myogenic markers

| | | | | | | |
|---|---|---|---|---|---|---|
| Desmin | nd | + | + | | | + |
| c-met | + | + | nd | + | + | + |
| Bcl-2 | nd | + | + | | | |
| myf5 | − | | | − | + | + |
| MNF | | + | + | | | + |

late myogenic markers

| | | | | | | |
|---|---|---|---|---|---|---|
| Myogenin | − | + | nd | − | + | |
| MyoD | − | + | + | − | + | + |

Hematopoetic stem cell marker

| | | | | | | |
|---|---|---|---|---|---|---|
| Flk-1 | − | + | nd | − | | |
| Sca-1 | ** | + | + | + | | |
| CD34 | +* | − | + | + | | +/−*** |
| m cadherin | | + | − | − | + | + |
| CD45 | − | − | − | − | | |

*CD34 is present at isolation PO and P3
** the human homologue of mouse sca-1 is not known
***CD 34 expression only during the first days of clonal growth.
**** − + refers to the absence of a marker after isolation and the presence of that marker after expansion and passaging of the cells.
(1) human SM MPC of the present invention
(2) mdx mice derived cell popluation mc13. Lee et al (1999) cited supra
(3) murine Muscle Derived Stem Cells Qu-petersen et al (2002) cited supra
(4) murine Interstitial Space of Skeletal Muscle derived cells. Tamaki et al (2002) J. Cell Biol. 157, 571-577.
(5) murine Embryonic Dorsal Aorta derived Skeletal Myogenic Precursor De Angelis et al (1999) J. Cell. Biol. 147, 869-877

SM-MPCs of the present invention have a clearly different expression pattern of molecular markers compared to muscle derived progenitors cell. Further, SM-MPC lack a number of myogenic markers. Only the freshly isolated precursor cells of muscle interstitial spaces derived cells show few myogenic markers. However after three days these cell are positive for every myogenic marker assayed.

No expression of MRFs by RT PCR was detected on human synovial tissue nor on SM-MPCs in monolayer either on tissue culture plastic or on gelatin-coated dishes, neither was myotube formation under low serum conditions at confluence observed. MRF expression and in vitro myotube formation are characteristic for myoblast cells (Gerhardt et al cited supra, Seale and Rudnicki cited supra, Seale et al cited supra).

SM-MPCs have a remarkable self renewal capacity and maintained a linear growth curve over at least 30 population doublings. Nevertheless, no telomerase activity was detected under our experimental conditions. This might be attributable to the length of the telomeres in the original cell population within the synovial tissue or by a telomerase-independent mechanism to preserve telomere length.

The SM-MPCs are also MyoD negative distinguishing them from fetal MyoD positive cells which can differentiate into skeletal muscle. The cells of the present invention were originally described as synovial membrane derived mesenchymal stem cells (SM-MSCs) (De Bari et al. cited supra). Human SM-MPCs have similarities to BM-MSCs in their in vitro behavior. Mesenchymal stem cells however do not express the CD34 cell marker (Pittenger et al, cited supra) while the SM-MPCs of the present invention do express CD34 after isolation and during expansion at P0 and in the cell population at P3. CD34 expression was encountered at least up to passage 8 by FACS analysis. This marker analysis clearly distinguishes the progenitor cells with in vivo myogenic potential from mesenchymal stem cells. SM-MPCs rapidly adhere to plastic and can be expanded for several passages, preserving their molecular profile and multipotentiality (De Bari et al. cited supra). These characteristics make MPCs, irrespective of their origin, quite distinct from Hematopoeitic Stem Cells (HSC) (Prockop cited supra; Pittenger et al. cited supra).

The possibility theoretically exist that in primary cultures of synovial membrane, some cells that would not have adhered to plastic may have remained associated with an underlying monolayer. Thereby, multipotent stem cells derived from the population of HSCs may have been maintained without a loss of myogenic potentiality and possibly expanded in the presence of a “feeder” layer. This possibility is however remote since single cells cloned by limiting dilution attached on plastic and grew in monolayer (De Bari et al. cited supra), displaying upon expansion myogenic potential both in vitro (De Bari et al. cited supra) and in vivo.

A possibility exists that SM-MPCs are derived from endogenous resident cells or that they might originate from circulating MSCs (Kuznetsov et al cited supra). Nevertheless, the derivation of SM-MPCs from circulating SMC populations would not exclude that, by residing in the SM, MSCs could acquire distinct biological properties. In addition, manipulations such as tissue dissection, cell isolation and subsequent culture expansion can profoundly influence patterns of gene expression and differentiation potentials, with as final result the generation of muscle progenitor cells from progenitor cells intended for the repair of the joint tissues.

The persistence in postnatal synovial tissue of cells with a phenotype reminiscent of the developing joint interzone (De Bari et al cited supra; Hartmann et al. cited supra) points to the SM as a possible reservoir of uncommitted progenitor cells for the repair of those joint tissues, such as articular cartilage and menisci, which have a limited capacity for intrinsic repair (Hunziker & Rosenbergcited supra).

### Example 8

### SM-MPCs in Nude Mice Model for Myocardial Infarction

Experimental Design:

SM-MPCs (between P3 and P10) are cultured in T75 flasks in DMEM complete (500 ml DMEM+56 ml FBS+5 ml antibiotics+5 ml sodiumpyruvate). At 70% confluence, cells are harvested, washed twice in serum free medium and 500.000 cells were injected (by 6 injections) into the cardiac muscle of nude mice in which a cardiac infarction has been induced (as described by Lutgens et al., 1999, Cardiovasc Res 41: 586-593). Negative controls were injected with DMEM only. At four different time points, mice are sacrificed and cardiac muscle is harvested for histology and total RNA extraction for RT-PCT. Controls are sacrificed at 2 and 4 weeks.

The functional impact of the cells on the heart muscle of the rats is evaluated at 2 and 4 weeks by means of echocardiogram, and at 4 weeks by electrocardiogram, and/or other function measurements.

Results:

At week 2, human cells were clearly detected to be present in the mouse cardiac muscle and subsequently were found to proliferate (based on human β-actin and β2-microglobulin expression). The marker NKx2.5 (human-specific), which was absent from the cells during cultivation, is detected with RT-PCR at week 2, indicating proper early differentiation. At week 2, echography also indicated the presence of newly formed tissue with indications of functional recovery of the infarcted myocardium.

1. A composition comprising a population of mammalian muscle progenitor cells derived from joint tissue, said cells having in vivo myogenic properties and providing a persistent pool of satellite cells when introduced into mammals and characterised by the expression of c-met as a positive marker or any marker coexpressed or co-detectable with this positive marker and by the expression of gdf5/cdmp1 as a negative marker or any marker coexpressed or co-detectable with this negative marker.

2-35. (canceled)

36. A composition according to claim 1 wherein the cells are derived from synovial membrane.

37. A composition according to claim 1 wherein the cell population is characterised by the expression of one or more of the synovial fibroblast positive markers CD44 and CD90 and by the absence of the expression of the negative markers flk-1 or any marker coexpressed or co-detectable with these positive and/or negative markers.

38. The composition according to claims 1 further characterised by the expression of CD34 as a positive marker or any marker coexpressed or co-detectable with this positive marker.

39. The composition according to claim 1 wherein the cells are genetically engineered.

40. The composition of claim 39 wherein the genetically engineered cells comprise a promoter operably linked to a nucleotide sequence encoding a protein selected from the group of an angiogenic factor, a peptide growth factor and an anti-angiogenic factor.

41. The composition according to claim 1 wherein the cells are clonal.

42. The composition according to claim 1 wherein the cells are isolated and passaged between 3 and 10 passages.

43. A pharmaceutical composition comprising a composition of muscle progenitor cells according to claim 1 in admixture with at least one pharmaceutically acceptable carrier.

44. A method for repairing or preventing muscle dysfunction in a patient, said method comprising administering the pharmaceutical composition of claim 9 to said patient.

45. The method of claim 44, wherein said dysfunction is selected from a severe trauma, a diffuse trauma and crush syndrome, disuse atrophy, sarcopenia.

46. The method of claim 44, wherein said muscle is cardiac muscle and said dysfunction is a cardiovascular disorder selected from myocardial infarct and heart failure.

47. A method for the restoration of Mechano Growth Factor expression by dystrophic muscle cells in a patient, said method comprising comprising administering the pharmaceutical composition of claim 43 to said patient.

48. A method of regenerating muscle comprising of the step of administrating a composition according to claim 1 to an individual.

49. The method of claim 48 wherein the composition is injected into the affected muscle.

50. A method of selecting muscle precursor cells comprising the step of simultaneously or subsequently contacting a joint tissue derived cell population with a binding substance for one or more of the positive marker c-Met and/or the negative marker and CDMP1 or any marker coexpressed or co-detectable with this positive or this negative marker.

51. The method according to claim 50 wherein the joint tissue derived cell population is obtained from the synovial membrane.

52. The method according to claim 50 wherein the binding substance is an antibody or a ligand for a receptor.

53. A method of providing a persistent reserve population of satellite cells in an individual comprising the step of administering a composition according to claim 1 to an individual.
